(19) United States
(12) Patent Application Publication
(10) Pub. No.: US 2007/0128142 A1
(43) Pub. Date: Jun. 7, 2007

(54) Emulsion for conveying a hydrophobic active material to a substrate in aqueous medium

(76) Inventors: Ian Harrison, Poissy (FR); Evelyne Prat, Pantin (FR); Helene Lannibois, Charenton Le Pont (FR); Anne-Gaelle Dreno, Sevran (FR)

Correspondence Address: Jean Louis Seugnet;Rhodia Inc, Legal Department CN 7500, 8 Cedar Brook Drive, Cranbury, NJ, 08512-7500, US

(21) Appl. No.: 10/579,512

(22) PCT Filed: Oct. 27, 2004
(86) PCT No.: PCT/FR04/02762

§ 371 Date: Jan. 22, 2007

(30) Foreign Application Priority Data
Nov. 13, 2003 (FR) 0313284
Jan. 23, 2004 (FR) 0400665

(51) Int. Cl. A61K 8/73; A61K 8/64; A61K 8/81

(52) U.S. Cl. 424/70.12; 424/70.13; 424/70.14; 424/70.16; 510/320

(57) ABSTRACT

Emulsion (E) comprising a hydrophobic phase (O) containing and/or consisting of a hydrophobic active material (A), which is in the form of a multiple emulsion (Em) comprising: an inner inverse emulsion (Ei) comprising the continuous phase (O), a dispersed aqueous phase (Wi) and, at the interface of the two phases, a stabilizer (Di) as a water-soluble or water-dispersible polysaccharide with a mean degree of polymerization of at least 1.5 and preferably of at least 20, the Brookfield viscosity of which, at 25° C. as a 1% solution in water, is less than 20 000 mPa·s, and free of lipophilic polyorganosiloxane substituting groups, and an aqueous or water-miscible outer phase (We), in which is dispersed the inner emulsion (Ei), or in a solid form (Es), which is water-dispersible as a multiple emulsion (Em); the emulsion (E), after being used in an aqueous medium (B), can convey the hydrophobic active material to a substrate present in or in contact with said aqueous medium (B).

The present invention relates to an emulsion (E), in the form of a multiple emulsion (Em), the inner inverse emulsion of which comprises a hydrophobic active material, or in solid form (Es) which is water-dispersible as a multiple emulsion of water-in-oil-in-water type (Em). This emulsion may be used to convey said hydrophobic active material to a substrate in aqueous medium. It may be used especially in a detergent or rinsing composition or as a detergent or rinsing composition for washing or rinsing articles made of textile fibers, for the purpose of conveying and promoting the deposition of a hydrophobic active material, for instance a fragrance, a hydrophobic care agent, etc., onto a textile article, especially made of cotton.

It has been proposed to use a multiple emulsion of water-in-oil-in-water (W/O/W) type to convey hydrophilic active materials.

Thus, EP-A-930 933 claims a process for the controlled release of an active principle contained in a multiple emulsion of water-in-oil-in-water type, characterized in that:
said multiple emulsion comprises an inverse emulsion Ei with an aqueous phase A1 that contains at least one hydrophilic active principle, said emulsion Ei being dispersed in the form of droplets of direct emulsion Ed, in an aqueous continuous phase A2, with the two emulsions Ed and Ei stabilized with at least one surfactant, which is present in the respective continuous phases thereof, and in that
said multiple emulsion is placed in contact with a sufficient amount of an agent, so as to convert it into a direct emulsion and to induce the release of the active principle contained in the aqueous phase A1 of the emulsion Ei, in the aqueous phase A2.
The surfactant present in the continuous aqueous phase A2 of the emulsion Ed is preferably chosen from water-soluble lecithins, sucrose esters, fatty acid esters, polyoxyethylenated alkylamides, triglyceride sulfates, alkyl sulfates, alkyl ether sulfates, alkyl sulfonates, alkylamine salts, fatty amines, lipoamino acids, alkylbetaines, alkylpolyglycol ethers, alkylene oxide copolymers, modified polyesters and silicone polymeric surfactants.
The surfactant present in the continuous phase of the emulsion Ei is preferably chosen from liposoluble lecithins, fatty acid esters of sorbitan, polyalkylene dipolyhydroxystearates, fatty acids, monoglycerides, polyglycerol esters, polyglyceryl polyricinoleate, and lactic and tartaric acid esters.
The continuous phase of the emulsion Ei is an oily phase composed of at least one oil chosen from mineral, plant and animal oils.
The direct emulsion Ed preferably comprises from 50% to 99% by volume of a continuous aqueous phase A2 per i to 50% of inverse emulsion Ei.
The inverse emulsion Ei is preferably composed of 50% to 99% by volume of a continuous phase per 1% to 50% of aqueous phase A1.

It is also known practice to prepare granules obtained by drying a multiple emulsion of water-in-oil-in-water type (W/O/W), the inner aqueous phase of which comprises a hydrophilic active material.

Thus, WO 02/32563 claims granules that can be obtained by drying an inverse emulsion dispersed in an outer aqueous phase,
(a) the inverse emulsion comprising an inner aqueous phase, comprising at least one hydrophilic active material dispersed in an inner oily phase, said inner oily phase comprising at least one nonionic or cationic surfactant and/or at least one nonionic or cationic amphiphilic polymer,
(b) the outer aqueous phase comprising:
   at least one nonionic polyoxyalkylenated surfactant and/or at least one nonionic polyoxyalkylenated amphiphilic polymer, and
   at least one water-soluble or water-dispersible compound chosen from (i) polymers obtained by polymerization of at least one carboxylic acid monomer, and of at least one ethylenically unsaturated monomer, and/or of at least one ethylenically unsaturated carboxylic acid polyoxyalkylenated ester; (ii) polymers derived from the polymerization of at least one carboxylic acid monomer and optionally comprising at least one hydrophobic graft; (iii) polypeptides of natural or synthetic origin, optionally comprising at least one hydrophobic graft; (iv) highly depolymerized polysaccharides optionally comprising at least one hydrophobic graft.
      These granules are redispersible in aqueous medium as a multiple emulsion.

The aim of the invention is an emulsion in the form of a multiple emulsion or in a dry form that can be redispersed as a multiple emulsion, the oil phase of which consists of or comprises a hydrophobic active material, this emulsion, after use in an aqueous medium, being capable of conveying said hydrophobic active material to a substrate present in or in contact with said aqueous medium.

A first subject of the invention consists of an emulsion (E) comprising a liquid or meltable hydrophobic phase (O) containing and/or consisting of at least one hydrophobic active material (A), said emulsion (E) being:
in the form of a multiple emulsion (Em) comprising:
   an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di)
   an aqueous or water-miscible outer phase (We), in which is dispersed the inner emulsion (Ei), by means of at least one dispersant and/or stabilizer (De)
or in a solid form (Es), which is water-dispersible as a multiple emulsion (Em) in which the outer phase (We) is aqueous, comprising
   the inverse emulsion (Ei) dispersed in a water-soluble or water-dispersible solid matrix (M), and
   the dispersant and/or stabilizer (De) located at the interface of the inverse emulsion (Ei) and of the matrix (M) and optionally dispersed in the matrix (M)
      said emulsion being characterized in that the stabilizer (Di) at the interface of the two phases of the inner inverse emulsion (Ei) is made of a material chosen from water-soluble or water-dispersible polysaccharides (PSA):
the mean degree of polymerization (DP) of which is at least 1.5, preferably at least 20 and most particularly at least 100,
the Brookfield viscosity of which, at 25° C. as a solution at 1% by mass in water, is less than 20 000 mPa·s and preferably less than 5000 mPa·s, most particularly ranging from 1 to 4500 mPa·s,
   said polysaccharides (PSA) also being free of lipophilic polyorganosiloxane substituent groups.
   Inverse Emulsion (Ei)

According to the invention, the hydrophobic phase (O) contains and/or consists of at least one hydrophobic active material (A).

The hydrophobic phase (O) and/or the active material (A) are made of at least one organic or organosilicon material or of a mixture of at least one organic material and of at least one organosilicon material, which is liquid or meltable and insoluble in an aqueous phase.

When the active material (A) is contained in the hydrophobic phase (O), it may also be made of at least one solid or liquid inorganic material that is insoluble in an aqueous phase or of a mixture of at least one inorganic material and of at least one organic material and/or of at least one organosilicon material.

According to the invention, the material constituting the hydrophobic phase (O) or the active material (A) is considered as insoluble when less than 15% and preferably less than 10% of its weight is soluble in the inner aqueous phase (Wi) and the outer phase (We) if the latter is present, this solubility applying over a temperature range of between 20° C. and the temperature of preparation of the inverse emulsion (Ei) and of the multiple emulsion (Em) or of the emulsion in a solid form (Es).

Said hydrophobic phase (O) preferably has a melting point of less than or equal to 100° C. and more particularly less than or equal to 80° C.

Said material constituting the hydrophobic phase (O) may also be made of an organosilicon material (O1).

It is in particular an oil, a wax or a resin made of a linear, cyclic, branched or crosslinked polyorganosiloxane.

Said polyorganosiloxane preferably has a dynamic viscosity, measured at 25° C. and at a shear rate of 0.01 Hz for a stress of 1500 Pa (performed on a Carrimed® CSL2-500 viscometer), of between 10⁴ and 10⁹ cP.

It may be especially:
a nonionic polyorganosiloxane
a polyorganosiloxane containing at least one cationic or potentially cationic function
a polyorganosiloxane containing at least one anionic or potentially anionic function
an amphoteric polyorganosiloxane containing at least one cationic or potentially cationic function and at least one anionic or potentially anionic function.

Preferably, it is a nonionic or amino polyorganosiloxane.

Examples of polyorganosiloxanes that may be mentioned include:
linear, cyclic or crosslinked polyorganosiloxanes formed from nonionic organosiloxane units of general formula
   (R)ₐ(X)_{b}Si(O)[4-(a+b)]/2 (I)
in which formula
the symbols R are identical or different and represent a linear or branched hydrocarbon-based alkyl radical containing from 1 to 4 carbon atoms or an aryl radical, especially phenyl;
the symbols X are identical or different and represent a hydroxyl group, a linear or branched alkoxy radical containing from 1 to 12 carbon atoms, or a function OCOR′, in which R′ represents an alkyl group containing from 1 to 12 carbon atoms and preferably 1 carbon atom;
a is equal to 0, 1,2 or 3
b is equal to 0, 1, 2 or 3
a+b is equal to 0, 1, 2 or 3.

Preferentially, said polyorganosiloxane is at least substantially linear and most preferentially linear. Examples that may especially be mentioned include α,ω-bis(hydroxy)polydimethylsiloxane oils, α,ω-bis(trimethyl)polydimethylsiloxane oils, cyclic polydimethylsiloxanes and polymethylphenylsiloxanes;
linear, cyclic or crosslinked polyorganosiloxanes comprising, per mole, at least one ionic or nonionic organosiloxane unit of general formula
   (R)ₐ(X)_{b}(B)_{c}Si(O)[4-(a+b+c)]/2 (II)
in which formula
the symbols R are identical or different and represent a linear or branched monovalent alkyl hydrocarbon-based radical containing from 1 to 4 carbon atoms, or an aryl radical, especially phenyl;
the symbols X are identical or different and represent a hydroxyl group, a linear or branched alkoxy radical containing from 1 to 12 carbon atoms, or a function OCOR′, in which R′ represents an alkyl group containing from 1 to 12 carbon atoms and preferably 1 carbon atom;
the symbols B are identical or different and represent an aliphatic and/or aromatic and/or cyclic hydrocarbon-based radical containing up to 30 carbon atoms, optionally interrupted with one or more oxygen and/or nitrogen and/or sulfur heteroatoms, optionally bearing one or more ether, ester, thiol, hydroxyl, optionally quaternized amine or carboxylate functions, the symbol B being linked to the silicon preferably via an Si—C— bond;
a is equal to 0, 1 or 2
b is equal to 0, 1 or 2
c is equal to 1 or 2
a+b+c is equal to 1, 2 or 3.

As examples of substituents corresponding to the symbol (B) in formula (II) above, mention may be made of
the polyether groups of formula
   —(CH₂)ₙ—(OC₂H₄)ₘ—(OC₃H₆)ₚ—OR′,
in which n is equal to 2 or 3, m and p each range from 0 to 30 and R′ represents an alkyl residue containing from 1 to 12 carbon atoms and preferably 1 to 4 carbon atoms.
primary, secondary, tertiary or quaternized amino groups, such as those of formula
   —R¹—N(R²)(R³)
in which
the symbol R′ represents an alkylene group containing from 2 to 6 carbon atoms, optionally substituted or interrupted with one or more nitrogen or oxygen atoms,
the symbols R² and R³, which may be identical or different, represent
   H,
   an alkyl or hydroxyalkyl group containing from 1 to 12 carbon atoms and preferably from 1 to 6 carbon atoms,
   an aminoalkyl group, which is preferably primary, the alkyl group of which contains from 1 to 12 carbon atoms and preferably from 1 to 6 carbon atoms, optionally substituted and/or interrupted with at least one nitrogen and/or oxygen atom,
said amino group being optionally quaternized, for example with a hydrohalic acid or an alkyl or aryl halide.

Mention may be made especially of those of formulae:
—(CH₂)₃NH₂ (CH₂)₃NH₃⁺X⁻
—(CH₂)₃N(CH₃)₂ (CH₂)₃N⁺(CH₃)₂(C₁₈H₃₇)X⁻
—(CH₂)₃NHCH₂CH₂NH₂ —(CH₂)₃N(CH₂CH₂OH)₂
—(CH₂)₃N(CH₂CH₂NH₂)₂

Preferentially, the polyorganosiloxanes bearing amino functions contain in their chain, per 100 silicon atoms in total, from 0.1 to 50, preferably from 0.3 to 10 and most particularly from 0.5 to 5 aminofunctionalized silicon atoms;
sterically hindered piperidinyl groups of formula III 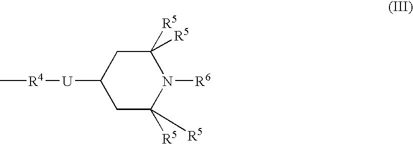
in which
   R⁴ is a divalent hydrocarbon-based radical chosen from:
      linear or branched alkylene radicals containing 2 to 18 carbon atoms;
      alkylenecarbonyl radicals in which the linear or branched alkylene portion contains 2 to 20 carbon atoms;
      alkylene-cyclohexylene radicals in which the linear or branched alkylene portion contains 2 to 12 carbon atoms and the cyclohexylene portion comprises an OH group and optionally 1 or 2 alkyl radicals containing 1 to 4 carbon atoms;
      radicals of formula —R⁷—O—R⁷ in which the radicals R⁷, which may be identical or different, represent alkylene radicals containing from 1 to 12 carbon atoms;
      the radicals of formula —R⁷—O—R⁷ in which the radicals R⁷ have the meanings given above and either or both of them is (are) substituted with one or two —OH group(s);
      the radicals of formula —R⁷—COO—R⁷ in which the radicals R⁷ have the meanings given above;
      the radicals of formula —R⁸—O—R⁹—O—CO—R⁸ in which the radicals R⁸ and R⁹, which may be identical or different, represent alkylene radicals containing 2 to 12 carbon atoms and the radical R⁹ is optionally substituted with a hydroxyl radical;
      U represents —O— or —NR¹⁰—, R¹⁰ being a radical chosen from a hydrogen atom, a linear or branched alkyl radical containing 1 to 6 carbon atoms and a divalent radical of formula: 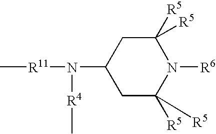
      in which R⁴ has the meaning given above, R⁵ and R⁶ have the meanings given below and R¹¹ represents a linear or branched divalent alkylene radical containing from 1 to 12 carbon atoms, one of the valency bonds (that of R¹¹) being linked to the atom of —NR¹⁰—, the other (that of R⁴) being linked to a silicon atom;
   the radicals R⁵ are identical or different and are chosen from linear or branched alkyl radicals containing 1 to 3 carbon atoms and a phenyl radical;
   the radical R⁶ represents a hydrogen radical or the radical R⁵ or O;
or the sterically hindered piperidyl groups of formula IV 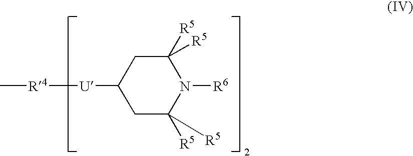
in which
   R′⁴ is chosen from a trivalent radical of formula: 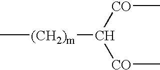
   which m represents a number from 2 to 20,
      and a trivalent radical of formula: 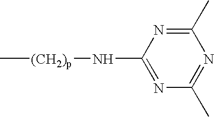
      in which p represents a number from 2 to 20;
   U′ represents —O— or NR¹²—, R¹² being a radical chosen from a hydrogen atom and a linear or branched alkyl radical containing 1 to 6 carbon atoms;
R⁵ and R⁶ have the same meanings as those given above for formula III.

Preferentially, said polyorganosiloxane containing a sterically hindered amine function is a linear, cyclic or three-dimensional polyorganosiloxane of formula (V): 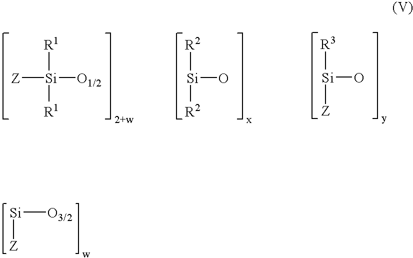
in which:
(1) the symbols Z, which may be identical or different, represent R¹ below or the symbol B below;
(2) the symbols R¹, R² and R³, which may be identical and/or different, represent a monovalent hydrocarbon-based radical chosen from linear or branched alkyl radicals containing from 1 to 4 carbon atoms, linear or branched alkoxy radicals containing from 1 to 4 carbon atoms, a phenyl radical and, preferably, a hydroxyl radical, an ethoxy radical, a methoxy radical or a methyl radical;
(3) the symbols B, functional groups which may be identical and/or different, represent a group containing sterically hindered piperidyl function(s) chosen from those mentioned above; and
(4) the number of organosiloxy units without a group B ranges from 10 to 450, preferably from 50 to 250;
   the number of organosiloxy units with a group B ranges from 1 to 5 and preferably from 1 to 3;
      0≦w≦10 and 8<y<448.

Most preferentially, said polyorganosiloxane is linear.

As examples of commercial polyorganosiloxane products that may be used as hydrophobic phase (A), mention may be made especially of the oils Rhodorsil® 21645 and Rhodorsil® Extrasoft sold by Rhodia.

Said material constituting the hydrophobic phase (O) may be made of an organic material (O2).

Examples that may be mentioned include:
C₁-C₃₀ carboxylic acid mono-, di- or triglycerides or mixtures thereof, for instance plant oils (rapeseed oil, castor oil, sunflower oil, erucic rapeseed oil, linseed oil, etc.)
technical oils, for instance the cooked, blown or standolized linseed oils sold by NOVANCE
sucroesters and sucroglycerides
C₁-C₃₀ alcohol esters of C₁-C₃₀ carboxylic acids or of C₂-C₃₀ dicarboxylic acids,
ethylene or propylene glycol monoesters or diesters of C₁-C₃₀ carboxylic acids,
propylene glycol C₄-C₂₀ alkyl ethers,
di(C₈-C₃₀)alkyl ethers,
mineral oils, for instance naphtha oils, liquid paraffins (petroleum jelly) and polybutenes,
organic waxes comprising alkyl chains containing from 4 to 40 carbon atoms. Among the waxes, mention may especially be made of:
   animal waxes (beeswax, lanolin or whale oil),
   plant waxes (carnauba wax, candelilla wax, sugar cane wax or jojoba wax),
   fossil mineral waxes (montan wax, ozokerite or Utah wax),
   hydrocarbon-based waxes containing from 4 to 35 carbon atoms (mineral oils, paraffins and microcrystalline waxes),
   synthetic waxes, for instance polyolefins (polyethylene or polypropylene), sterone and carbowax.
      The active material (A) may constitute the hydrophobic phase (O) or may be contained in the hydrophobic phase (O); when it is contained in the hydrophobic phase (O) it may be soluble, partially soluble or insoluble in said hydrophobic phase (O).

As active materials (A) contained in the hydrophobic phase (O), mention may be made of other hydrophobic materials such as, especially, fragrancing molecules, organic or organosilicon UV stabilizers, hydrophobic bactericides, solid polyamide capsules, silica particles or other mineral oxides or compounds, etc.

Stabilizer (Di)

The stabilizer (Di) is made of a material chosen from water-soluble or water-dispersible polysaccharides (PSA):
the mean degree of polymerization (DP) of which is at least 1.5, preferably at least 20 and most particularly at least 100,
the Brookfield viscosity of which, at 25° C. as a solution at 1% by mass in water, is less than 20 000 mPa·s and preferably less than 5000 mPa·s, most particularly ranging from 30 to 4500 mPa·s.,
   said polysaccharides (PSA) also being free of lipophilic polyorganosiloxane substituent groups.

The term “water-soluble or water-dispersible” means herein that said polysaccharide (PSA) is incapable of forming a macroscopic two-phase solution at 25° C. in an inner aqueous phase (Wi).

The term “lipophilic” is used herein asynonymously with the term “hydrophilic”; i.e. having no affinity for water; this means that the polyorganosiloxane groups, of which the polysaccharide (PSA) is free, would form, by themselves, at a concentration of 10% by weight, a macroscopic two-phase solution in distilled water at 25° C. According to the invention, the Brookfield viscosity at 25° C. of a solution containing 1% by mass of polysaccharide (PSA) in an aqueous solution containing 1% by volume of HCl may be determined as follows, after maturation for 24 hours at 40° C.

Materials and Reagents

Materials
800 ml low-sided beaker
250 ml flask
stirring motor for obtaining 500 rpm, equipped with a Rayneri deflocculating paddle 65 mm in diameter
Brookfield LVT viscometer equipped with a No. 2 needle (planetary or disk type)
oven set at 40° C.

Reagents
distilled water
hydrochloric acid solution at 10% by volume. Introduce 27.0 g of analytical grade 37% hydrochloric acid (density=1.19) into a 100 ml flask containing about 50 ml of distilled water. Make up to 100 ml with distilled water.
   Procedure
weigh out 0.9 g of polysaccharide on a sheet of glazed paper
weigh out 269.1 g of water in an 800 ml beaker
start stirring the water and then add the powder over 20 to 30 seconds on the edges of the vortex. Stir for 30 minutes at 500 rpm.
While stirring, introduce into the solution 30 g of the 10% hydrochloric acid solution over 5 to 10 seconds. Stir for a further 5 minutes at 500 rpm
Transfer the solution into the 250 ml flask and place it in the oven at 40° C.
After 24 hours, remove the flask from the oven and return the temperature of the solution to room temperature (20-25° C.).
Measure the viscosity of the solution with the Brookfield viscometer set at 3 rpm and equipped with a No. 2 needle (planetary). Take the reading after stabilizing the display (N).
   Expressing the Results

Apparent viscosity η of the solution containing 1% polysaccharide (PSA) in the solution containing 1% HCl
η=100×N

The term “mean degree of polymerization” means the mean number of glycosyl units per mole of polysaccharide.

Said polysaccharide (PSA) is a homopolysaccharide or a heteropolysaccharide; it may be linear or branched, and nonionic or ionic; it may optionally be substituted and/or modified with nonionic or ionic groups, other than lipophilic polyorganosiloxane groups. Preferentially, said polysaccharide (PSA), or its skeleton, comprises identical or different glycosyl units linked together via β(1-4) bonds. It may also comprise, along with the β(1-4) bonds, other bonds, especially β(1-3) and/or β(1-6) bonds.

Said identical or different glycosyl units may especially be hexose and/or pentose units.

Among the hexose units (identical or different), mention may be made especially of D-glucose, D- or L-galactose, D-mannose, D- or L-fucose and L-rhamnose etc. units. Among the pentose units (identical or different), mention may be made especially of D-xylose, L- or D-arabinose, etc. units.

The hydroxyl function(s) of the glycosyl units may be modified and/or substituted with nonionic, ionic or ionizable groups.

When such groups are nonionic modifying groups, they may especially be linked to the carbon atoms of the sugar skeleton either directly or via —O— bonds.

Among the non-ionic groups, mention may be made of:
alkyl groups containing from 1 to 22 carbon atoms, optionally interrupted with one or more oxygen and/or nitrogen heteroatoms,
aryl or arylalkyl groups containing from 6 to 12 carbon atoms,
hydroxyalkyl or cyanoalkyl groups containing from 1 to 6 carbon atoms,
“ester” groups obtained by replacing the hydrogen of a hydroxyl function —OH of the polysaccharide skeleton with a group comprising at least one acid unit especially containing carbon, sulfur or phosphorus, such as, especially, carbonyl R—(CO)—, sulfonyl R—SO₂—, phosphoryl R₂P(O)— and hydroxyphosphoryl R—P(O)(OH)— groups, the acid groups forming “ester” units with the remaining oxygen atoms of the polysaccharide skeleton. The alkyl, alkenyl or aryl group R may contain from 1 to 20 carbon atoms; it may also comprise a heteroatom, for example nitrogen, directly linked to a carbonyl, sulfonyl, etc. unit and thus form bonds of the urethane, etc. type.

Examples that may be mentioned include:
methyl, ethyl, propyl, isopropyl, butyl, hexyl, octyl, dodecyl, octadecyl and phenyl groups, linked to a carbon atom of the polysaccharide skeleton via an ether, ester, amide or urethane bond,
cyanoethyl, hydroxyethyl, hydroxypropyl or hydroxybutyl groups, linked to a carbon atom of the polysaccharide skeleton via an —O— bond,
“ester” groups chosen from acetate, propanoate, trifluoroacetate, 2-(2-hydroxy-1-oxopropoxy)propanoate, lactate, glycolate, pyruvate, crotonate, isovalerate, cinnamate, formate, salicylate, carbamate, methylcarbamate, benzoate, gluconate, methanesulfonate and toluenesulfonate groups; hemiester groups of fumaric, malonic, itaconic, oxalic, maleic, succinic, tartaric, aspartic, glutamic or malic acid; mention may be made more particularly of acetate, hemiacetate and 2-(2-hydroxy-1-oxopropoxy)propanoate substituent groups.

The degree of modification MS of a polysaccharide with a nonionic modifying group corresponds to the mean number of moles of precursor of the nonionic modifying group that have reacted per glycosyl unit.

The degree of modification MS can vary according to the nature of the precursor of said modifying group.

If said precursor is incapable of forming novel reactive hydroxyl groups (for example alkylation precursor), the degree of modification with the nonionic groups is less than 3 by definition.

If said precursor is capable of forming novel reactive hydroxyl groups (for example hydroxyalkylation precursor), the degree of modification MS is theoretically unlimited; it may be, for example, up to 6 and preferably up to 2. This degree is generally at least 0.001 and preferably at least 0.01.

Among the anionic or anionizable groups, mention may be made of those containing one or more carboxylate, sulfonate, sulfate, phosphate, phosphonate, etc. functions.

Mention may be made in particular of those of formula:
—[—CH₂—CH(R)—O]ₓ—(CH₂)_{y}—COOH or
—[—CH₂—CH(R)—O]ₓ—(CH₂)_{y}—COOM, in which
   R is a hydrogen atom or an alkyl radical containing from 1 to 4 carbon atoms
   x is an integer ranging from 0 to 5
   y is an integer ranging from 0 to 5
   M represents an alkali metal.

Mention may be made most particularly of carboxy groups —COO⁻Na⁺ linked directly to a carbon atom of the sugar skeleton, carboxymethyl groups (sodium salt) —CH₂—COO^{-Na}⁺ linked to a carbon atom of the sugar skeleton via an —O— bond.

Among the cationic or potentially cationic groups, mention may be made of those containing one or more amino, ammonium, phosphonium, pyridinium, etc. functions.

Mention may be made in particular of the cationic or potentially cationic groups of formula
—NH₂
—[—CH₂—CH(R)—O]ₓ—(CH₂)_{y}—COA-R′—N(R″)₂
[—CH₂—CH(R)—O]ₓ—(CH₂)_{y}—COA-R′—N⁺(R′″)₃ X—
—[—CH₂—CH(R)—O]ₓ—(CH₂)_{y}—COA-R′—NH—R″″—N(R″)₂
—[—CH₂—CH(R)—O]ₓ—R′—N(R″)₂
—[—CH₂—CH(R)—O]ₓ—R′—N+(R″″)₃ X—
—[—CH₂—CH(R)—O]ₓ—R′—NH—R″″—N(R′)₂
—[—CH₂—CH(R)—O]ₓ—Y—R″
   in which.
   R is a hydrogen atom or an alkyl radical containing from 1 to 4 carbon atoms
   x is an integer ranging from 0 to 5
   y is an integer ranging from 0 to 5
   R′ is an alkylene radical containing from 1 to 12 carbon atoms, optionally bearing one or more OH substituents
   the radicals R″, which may be identical or different, represent a hydrogen atom or an alkyl radical containing from 1 to 18 carbon atoms
   the radicals R′″, which may be identical or different, represent an alkyl radical containing from 1 to 18 carbon atoms
   R″″ is a linear, branched or cyclic alkylene radical containing from 1 to 6 carbon atoms
   A represents O or NH
   Y is a heterocyclic aliphatic group containing from 5 to 20 carbon atoms and a nitrogen heteroatom
   X— is a counterion, preferably a halide (especially chloride, bromide or iodide), and also N-alkylpyridiniumyl groups in which the alkyl radical contains from 1 to 18 carbon atoms, with a counterion, preferably a halide (especially chloride, bromide or iodide).

Among the cationic or potentially cationic groups, mention may be made most particularly of:
those of formula
—NH₂
—CH₂—CONH—(CH₂)₂—N(CH₃)₂
—CH₂—COO—(CH₂)₂—NH—(CH₂)₂—N(CH₃)₂
—CH₂—CONH—(CH₂)₃—NH—(CH₂)₂—N(CH₃)₂
—CH₂—CONH—(CH₂)₂—NH—(CH₂)₂—N(CH₃)₂
—CH₂—CONH—(CH₂)₂—N⁺(CH₃)₃ Cl⁻
—CH₂—CONH—(CH₂)₃—N⁺(CH₃)₃ Cl⁻
—(CH₂)₂—N(CH₃)₂
—(CH₂)₂—NH—(CH₂)₂—N(CH₃)₂
—(CH₂)₂—N⁺(CH₃)₃ Cl⁻
most preferentially 2-hydroxypropyltrimethylammonium chloride
   —CH₂—CH(OH)—CH₂—N⁺(CH₃)₃ Cl⁻
pyridiniumyl groups such as N-methylpyridiniumyl, of formula 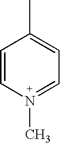
with a chloride counterion
hindered amino groups such as those derived from HALS amines, of general formula: 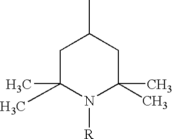
in which R represents CH₃ or H.

Among the betaine groups, mention may be made most particularly of the functions of formula: 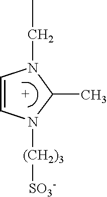
2-methyl(3-sulfopropyl)imidazolium function 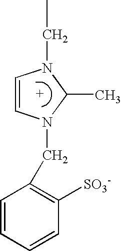
(2-sulfobenzyl)imidazolium function 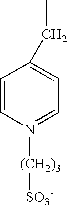
(3-sulfopropyl)pyridinium function —(CH₂)₂—N⁺(CH₃)₂—(CH₂)₂—COO⁻ ethyldimethylammonium betaine function —(CH₂)₂—N⁺(CH₃)₂—(CH₂)₃—SO₃⁻ sulfopropyldimethylammonium function.

The degree of substitution DS corresponds to the mean number of hydroxyl functions of the glycosyl units substituted with said ionic or ionizable group(s), per glycosyl unit.

It is generally less than 3 and preferably less than 2.

Among the polysaccharides (PSA) that may be used according to the invention, mention may be made of natural or synthetic polysaccharides, which are preferably chemically modified and/or substituted and/or degraded (depolymerized) via acidic or basic hydrolysis, or via an oxidative, thermal or enzymatic route.

Examples that may be mentioned include:

Galactomannans (especially guar gum), which are depolymerized, especially oxidatively, optionally modified or substituted with nonionic groups (especially hydroxypropyl), anionic groups (especially carboxymethyl) or cationic groups (for example hydroxypropyltrimethylammonium chloride), having a Brookfield viscosity (1% solution in water) that may range from 30 to 4500 mPa·s and preferably from 60 to 3500 mPa·s.

Mention may be made especially of:
Oxidatively depolymerized guars (containing a few COOH⁺ functions resulting from depolymerization in oxidizing medium), for instance the products Meypro-Gat 7, Meypro-Gat 20 and Meypro-Gat 30 sold by Rhodia
hydroxypropyl depolymerized guars with a degree of modification of about from 0.01 to 0.8, for instance the product HMP-CON from Rhodia
carboxymethyl depolymerized guars with a degree of substitution of about from 0.05 to 1.6, for instance the product Meypro-Gum R 600 sold by Rhodia
cationized depolymerized guars with a degree of substitution of about from 0.04 to 0.17 and preferably from 0.06 to 0.14, for instance the product Meypro-Coat 21 sold by Rhodia or AquaCat CG 518 sold by Aqualon.

Modified celluloses, for instance
cellulose monoacetates, with a degree of substitution of from 0.3 to less than 1.2 and preferably from 0.3 to 1
hydroxypropylcelluloses with a degree of modification of about from 0.2 to 1.5, for instance the product Primaflo HP22 sold by Aqualon
carboxymethylcelluloses with a degree of substitution of from 0.05 to 1.2 and preferably from 0.05 to 1, for instance Blanose Cellulose Gum from Hercules and Liberty 3794 from Aqualon

Dextrins optionally containing hydroxyethyl, hydroxypropyl or quaternized aminoalkyl groups (degradation of starches, optionally chemically modified with hydroxyethyl, hydroxypropyl or quaternized aminoalkyl groups).
Xyloglycans, for instance the tamarind gum Instasol 1200 from Saiguru Food, or Meypro-Gum T12 sold by Rhodia.
Arabinoxylans
Alkylpolyglycosides (APG) containing a C₄-C₂₀ and preferably C₈-C₁₈ alkyl group, and also a mean number of glucose units of about 1.5 to 10, preferably from about 1.5 to 4 and more generally from about 1.6 to 2.7 per mole of alkylpolyglycoside (APG), for instance those mentioned in U.S. Pat. No. 4,565,647.
   Inner Aqueous Phase (Wi)

The mean size of the aqueous droplets (Wi) of the inner inverse emulsion (Ei) may range up to 10 μm, preferably from 0.05 μm to 5 μm and more preferentially from 0.1 to 1 μm.

The mean size corresponds to the median volume diameter (d50), which represents the diameter of the particle equal to 50% of the cumulative distribution; it may be measured, for example, with an Horiba granulometer or an optical microscope.

The dispersed aqueous phase (Wi) has a pH that may range from 0 to 14, preferably from 2 to 11 and more preferentially from 5 to 11.

It may contain additives for adjusting the osmotic pressure, for instance salts (sodium chloride or sulfate, calcium chloride, etc.) or sugars (glucose) or polysaccharides (dextran, etc.).

It may also contain buffers, hydrophilic active materials, especially antibacterial agents, for instance methylchloroisothiazolinone and methylisothiazolinone (Kathon® CG sold by Rohm & Haas), other water-soluble or water-dispersible materials, and also hydrophobic materials that are insoluble in the hydrophobic phase (O).

For good implementation of the invention, the mass ratio of the dispersed aqueous phase (Wi) to the hydrophobic phase (O) may range from 5/95 to 95/5 and preferably from 30/70 to 80/20.

The ratio of the mass of stabilizer (Di) to the mass of hydrophobic phase (O) may range from 0.1/100 to 500/100, preferably from 0.5/100 to 100/100 and most particularly from 0.5/100 to 50/100.

The inverse emulsion (Ei) may be obtained in a conventional manner.

For example, it may be obtained by dissolving and/or dispersing the polysaccharide (PSA) in water, followed by adding the aqueous solution and/or dispersion obtained to the hydrophobic phase (O), with stirring.

The stirring may advantageously be performed using a frame paddle, a mixer of planetary type, or a mixer containing a mobile doctor blade and a paddle rotating in opposite directions (counter-stirring).

The preparation of the inverse emulsion is generally performed at a temperature above the melting point of the material used as hydrophobic phase, but below the degradation point of the components included in the composition of the inverse emulsion. More particularly, this temperature is between 10 and 80° C.

The stirring time may be determined without difficulty by a person skilled in the art. It is preferably sufficient to obtain a mean size of aqueous droplets of the inverse emulsion of less than 10 μm, as mentioned above.

The amounts of the various constituents of the inverse emulsion (Ei) have already been defined above.

Multiple Emulsion (Em)

The multiple emulsion (Em) comprises the inverse emulsion (Ei) above, as inner emulsion, dispersed in an aqueous or water-miscible outer phase (We), comprising at least one dispersant and/or stabilizer (De).

Said dispersant and/or stabilizer (De) has a hydrophilic tendency. Preferentially, said dispersant and/or stabilizer (De) is chosen from hydrophilic surfactants and/or hydrophilic polymers and/or amphiphilic hydrophilic polymers. The term “hydrophilic” is used, in its usual sense, to mean “having affinity for water”; this means that the dispersant and/or stabilizer (De) is incapable of forming a macroscopic two-phase solution in distilled water at 25° C.

Preferentially, the outer phase (We) is an aqueous phase.

More particularly, the surfactants and/or polymers (De) satisfy the Bancroft rule and are preferably chosen from compounds that comply with both of the conditions below:
when they are mixed with the outer aqueous phase at a concentration of between 0.1 and 10% by weight of said phase and between 20 and 30° C., they are in the form of a solution in all or part of the concentration range indicated,
when the are mixed with the inner hydrophobic phase (O) at a concentration of between 0.1% and 10% by weight of said phase and between 20 and 30° C., they are in the form of a dispersion in all or part of the concentration range indicated.

For good implementation of the invention, said dispersant and/or stabilizer (De) may be formed from
(a) at least one nonionic hydrophilic surfactant
(b) at least one anionic hydrophilic surfactant
(c) at least one cationic hydrophilic surfactant
(d) at least one nonionic hydrophilic polymer
(e) at least one nonionic hydrophilic amphiphilic polymer
(f) at least one anionic hydrophilic polymer
(g) at least one anionic hydrophilic amphiphilic polymer
(h) at least one cationic hydrophilic polymer
(i) at least one cationic hydrophilic amphiphilic polymer
(j) or a mixture of at least two of said compatible surfactants and/or polymers (a) to (d) above.

The term “compatible” means herein that the surfactants mixed together are incapable of leading to the formation of aggregates in non-dispersed form in the medium under consideration.

The total content of surfactant(s) and/or polymer(s) (De) present in the outer phase (We) may be between 0.01% and 50% by weight, preferably between 0.1% and 10% and more particularly between 0.5% and 5% by weight relative to the inverse emulsion (Ei).

Preferentially, the nonionic hydrophilic surfactants have an HLB value at least equal to the required HLB of the hydrophobic phase (O); as a guide, this minimum HLB value is usually at least 10. These surfactants are preferably polyalkoxylated. Advantageously, the polyalkoxylated nonionic surfactant may be chosen from the following surfactants, alone or as a mixture:
alkoxylated fatty alcohols
alkoxylated mono-, di- and triglycerides
alkoxylated fatty acids
alkoxylated sorbitan esters
alkoxylated fatty amines
alkoxylated bis(1-phenylethyl)phenols
alkoxylated tris(1-phenylethyl)phenols
alkoxylated alkylphenols more particularly ethoxylated and/or propoxylated.

Purely for illustrative purposes, the total number of ethoxylated and optionally propoxylated units is between 10 and 100.

Among the anionic hydrophilic surfactants, mention may be made of: alkyl ester sulfonates,

alkyl sulfates, and also polyalkoxylated (ethoxylated (EO) or propoxylated (PO), or combinations thereof) derivatives thereof, for instance sodium dodecyl sulfate;
alkyl ether sulfates, and also polyalkoxylated (ethoxylated (EO) or propoxylated (PO), or combinations thereof derivatives thereof, for instance lauryl ether sulfate with n=2;
alkylamide sulfates, and also polyalkoxylated (ethoxylated (EO) or propoxylated (PO), or combinations thereof derivatives thereof;
saturated or unsaturated fatty acid salts, and
alkyl and/or alkyl ether and/or alkylaryl ether phosphate esters.

Among the cationic hydrophilic surfactants that may also be present in the dispersant and/or stabilizer (De), mention may be made of the quaternary ammonium salts of formula R¹R²R³R⁴N⁺X⁻ in which the groups R are long or short alkyl, hydroxyalkyl or ethoxylated alkyl hydrocarbon-based chains, X being a counterion (R¹ is a C₈-C₂₂, preferably C₈-C₁₀, or C₁₂-C₁₄ alkyl group and R² is a methyl group, R³ and R⁴, which may be identical or different, being a methyl or hydroxymethyl group); and also cationic esters, for instance choline esters.

As regards the nonionic hydrophilic polymers that may be used, mention may be made of the nonionic water-soluble or water-dispersible polysaccharides (PSA) already mentioned above as stabilizer (Di), for instance
hydroxypropylcelluloses with a degree of modification of about from 0.2 to 1.5, or
cellulose monoacetates with a degree of substitution of from 0.3 to less than 1.2.

As regards the nonionic hydrophilic amphiphilic polymers, they are preferably polyalkoxylated compounds, comprising at least two blocks, one of them being hydrophilic, the other hydrophobic, at least one of the blocks comprising polyalkoxylated units, more particularly polyethoxylated and/or polypropoxylated units.

More particularly, said nonionic polyalkoxylated hydrophilic amphiphilic polymers are chosen from polymers with a weight-average molar mass of less than or equal to 100 000 g/mol (measured by GPC, with a polyethylene glycol standard), preferably between 1000 and 50 000 g/mol and preferably between 1000 and 20 000 g/mol.

As examples of polymers of this type, mention may be made, inter alia, of polyethylene glycol/polypropylene glycol/polyethylene glycol triblock polymers. Such polymers are well known and are especially sold under the brand names Pluronic (sold by BASF) and Arlatone (sold by ICI).

Said nonionic hydrophilic amphiphilic polymer may also be a block hydrophilic amphiphilic polymer, obtained by polymerizing at least one nonionic hydrophilic monomer and at least one hydrophobic monomer, the proportion and nature of said monomers being such that the resulting polymer satisfies the conditions stated previously (Bancroft rule—two conditions). They comprise at least one hydrophobic block and at least one neutral (nonionic) hydrophilic block. When said polymer comprises at least three blocks, and more particularly three blocks, the polymer is advantageously linear. In addition, the hydrophilic blocks are found more particularly at the ends.

When the polymers comprise more than three blocks, these polymers are preferably in the form of grafted or comb polymers.

Among the hydrophobic monomers from which the hydrophobic block(s) of the hydrophilic amphiphilic polymer may be prepared, mention may be made especially of:
esters of linear, branched, cyclic or aromatic monocarboxylic or polycarboxylic acids comprising at least one ethylenic unsaturation,
esters of saturated carboxylic acids containing 8 to 30 carbon atoms, optionally bearing a hydroxyl group;
α,β-ethylenically unsaturated nitriles, vinyl ethers, vinyl esters, vinylaromatic monomers and vinyl or vinylidene halides,
linear or branched, aromatic or nonaromatic hydrocarbon-based monomers comprising at least one ethylenic unsaturation,
monomers of cyclic or noncyclic siloxane type, and chlorosilanes;
propylene oxide or butylene oxide;
alone or as mixtures, and also macromonomers derived from such monomers.

Particular examples that may be mentioned include:
(meth)acrylic acid esters with an alcohol containing 1 to 12 carbon atoms, for instance methyl (meth)acrylate, ethyl (meth)acrylate, propyl (meth)acrylate, n-butyl (meth)acrylate, t-butyl (meth)acrylate, isobutyl (meth)acrylate or 2-ethylhexyl acrylate;
vinyl acetate, vinyl Versatate®, vinyl propionate, vinyl chloride, vinylidene chloride, methyl vinyl ether or ethyl vinyl ether;
vinyl nitriles more particularly including those containing from 3 to 12 carbon atoms such as, in particular, acrylonitrile and methacrylonitrile;
styrene, α-methylstyrene, vinyltoluene, butadiene and chloroprene;
   alone or as mixtures, and also macromonomers derived from such monomers.

The preferred monomers are acrylic acid esters with linear or branched C₁-C₄ alcohols such as methyl acrylate, ethyl acrylate, propyl acrylate and butyl acrylate, vinyl esters, for instance vinyl acetate, styrene and α-methylstyrene.

As regards the nonionic hydrophilic monomers from which the block hydrophilic amphiphilic polymers may be obtained, mention may be made of ethylene oxide; linear, branched, cyclic or aromatic monocarboxylic or polycarboxylic acid amides, comprising at least one ethylenic unsaturation, or derivatives, for instance (meth)acrylamide or N-methylol(meth)acrylamide; hydrophilic esters derived from (meth)acrylic acid, for instance 2-hydroxyethyl (meth)acrylate; vinyl esters that allow polyvinyl alcohol blocks to be obtained after hydrolysis, for instance vinyl acetate, vinyl Versatate® or vinyl propionate. These monomers may be used alone or in combination, and also in the form of macromonomers. It is recalled that the term “macromonomer” denotes a macromolecule bearing one or more functions that are polymerizable via the method used.

The preferred hydrophilic monomers are acrylamide and methacrylamide, alone or as a mixture, or in the form of macromonomers.

Preferentially, the nonionic hydrophilic amphiphilic polymers have an HLB value at least equal to the required HLB of the hydrophobic phase (O); as a guide, this minimum HLB value is usually at least 10.

Among the anionic hydrophilic polymers that may be used, mention may be made of the anionic water-soluble or water-dispersible polysaccharides (PSA) already mentioned above as stabilizer (Di), for instance:
oxidatively depolymerized guars (containing a few COOH⁺ functions resulting from depolymerization in oxidizing medium), for instance the products Meypro-gat 7, Meypro-gat 20 and Meypro-gat 30 sold by Rhodia hydroxypropyl depolymerized guars with a degree of modification of about from 0.01 to 0.8, for instance the product HMP-CON from Rhodia
carboxymethyl depolymerized guars with a degree of substitution of about from 0.05 to 1.6, for instance the product Meypro-gum R 600 sold by Rhodia.

Among the anionic hydrophilic amphiphilic polymers that may be used, mention may be made most particularly of block polymers, preferably diblock or triblock polymers, obtained by polymerization of at least one anionic hydrophilic monomer, optionally of at least one nonionic hydrophilic monomer, and of at least one hydrophobic monomer.

In this case also, the choice of monomers and their respective proportions are such that the resulting polymer satisfies the two conditions mentioned previously (Bancroft rule).

As regards the anionic hydrophilic monomers from which the block hydrophilic amphiphilic polymers may be obtained, mention may be made of:
linear, branched, cyclic or aromatic monocarboxylic or polycarboxylic acids, and N-substituted derivatives of such acids; polycarboxylic acid monoesters, comprising at least one ethylenic unsaturation;
linear, branched, cyclic or aromatic vinylcarboxylic acids;
amino acids comprising one or more ethylenic unsaturations; alone or as mixtures, precursors thereof, sulfonic or phosphonic derivatives thereof, and also macromonomers derived from such monomers; the monomers or macromonomers possibly being in the form of salts.

Mention may be made more particularly of:
acrylic acid, methacrylic acid, fumaric acid, itaconic acid, citraconic acid, maleic acid, acrylamidoglycolic acid, 2-propene-1-sulfonic acid, methallylsulfonic acid, styrenesulfonic acid, a-acrylamidomethylpropanesulfonic acid, 2-sulfoethylene methacrylate, sulfopropylacrylic acid, bis(sulfopropyl)acrylic acid, bis(sulfopropyl)methacrylic acid, sulfatoethylmethacrylic acid, the phosphate monoester of hydroxyethylmethacrylic acid, and also the alkali metal salts, for instance the sodium or potassium salts, or the ammonium salts;
vinylsulfonic acid, vinylbenzenesulfonic acid, vinylphosphonic acid, vinylidenephosphoric acid and vinylbenzoic acid, and also the alkali metal salts, for instance the sodium or potassium salts, or the ammonium salts;
N-methacryloylalanine or N-acryloylhydroxyglycine; alone or as mixtures, and also macromonomers derived from such monomers.

Examples of nonionic hydrophilic monomers and of hydrophobic monomers have already been mentioned above.

Among the cationic hydrophilic polymers that may be used, mention may be made of the cationic water-soluble or water-dispersible polysaccharides (PSA) already mentioned above as stabilizer (Di), for instance:
cationized depolymerized guars with a degree of substitution of about from 0.04 to 0.17 and preferably from 0.06 to 0.14, for instance the product Meypro-coat 21 sold by Rhodia or AquaCat CG 518 sold by Aqualon.

According to a first preferential embodiment of the invention, the dispersant and/or stabilizer (De) is chosen from nonionic hydrophilic surfactants and/or nonionic hydrophilic (amphiphilic) polymers; more preferentially, said nonionic hydrophilic surfactants and/or nonionic hydrophilic (amphiphilic) polymers have an HLB value at least equal to the required HLB of the hydrophobic phase (O); as a guide, this minimum HLB value is usually at least 10.

The hydrophilic amphiphilic agent (De) is most particularly a nonionic hydrophilic amphiphilic polymer.

According to a second preferential embodiment of the invention, the dispersant and/or stabilizer (De) is a mixture of at least one nonionic hydrophilic surfactant and of at least one anionic hydrophilic surfactant, optionally combined with at least one nonionic hydrophilic (amphiphilic) polymer.

According to a third embodiment of the invention, the dispersant and/or stabilizer (De) is chosen from cationic hydrophilic surfactants and/or cationic hydrophilic (amphiphilic) polymers optionally mixed with at least one nonionic hydrophilic surfactant and/or at least one nonionic hydrophilic (amphiphilic) polymer. When a nonionic surfactant and/or a nonionic polymer is present, it may represent up to 50% of the weight of the cationic/nonionic mixture.

According to a fourth embodiment, the dispersant and/or stabilizer (De) is chosen from stabilizers (Di) made of at least one nonionic, anionic or cationic water-soluble or water-dispersible polysaccharide (PSA), or compatible mixtures thereof. At least one other compatible dispersant and/or stabilizer (De) may optionally be added thereto; preferentially, the amount by mass of dispersant and/or stabilizer (De) other than water-soluble or water-dispersible polysaccharide (PSA), which is optional, is not more than the mass of water-soluble or water-dispersible polysaccharide (PSA) used as dispersant and/or stabilizer (De).

The amount of outer phase (We) of the multiple emulsion (Em) depends on the concentration desired for the multiple emulsion (Em).

The mass ratio of inner inverse emulsion (Ei)/outer phase (We) comprising the dispersant and/or stabilizer (De) may range from 50/50 to 99/1, preferably from 70/30 to 98/2 and most particularly from 70/30 to 80/20.

The mass ratio, expressed as solids, of dispersant and/or stabilizer (De)/mass of the inner inverse emulsion (Ei) may range from 0.01/100 to 50/100, preferably from 0.1/100 to 10/100 and most particularly from 0.5/100 to 5/100.

The concentration of dispersant and/or stabilizer (De) in the outer phase (We) may range from 1% to 50%.

Preferentially, the emulsion (E) is a multiple emulsion (Em) comprising at least 70% by weight of inner emulsion (Ei).

The mean size of the globules of inner inverse emulsion (Ei) dispersed in the outer phase (We) is preferably less than 200 μm; it may preferably range from 1 to 20 μm and more preferentially from 5 to 15 μm.

For good implementation of the invention, the mean size of the globules of inner inverse emulsion (Ei) dispersed in the outer phase (We) is at least twice, preferably at least 5 times and most particularly at least 10 times greater than the mean size of the droplets of the inner aqueous phase (Wi) dispersed in the hydrophobic phase.

The outer phase (We) may be an aqueous or a water-miscible nonaqueous phase. The term “water-miscible” means that it is incapable of forming a macroscopic two-phase solution at 25° C.

When the outer phase (We) is a water-miscible nonaqueous phase, it is especially an alcoholic or aqueous-alcoholic phase, in particular a phase based on isopropyl alcohol, ethanol, etc.

Preferentially, the outer phase (We) is an aqueous phase.

The pH of the outer aqueous phase is not limiting; it depends on the intended application of the invention. The pH may range from 0 to 14, preferably from 2 to 11 and more preferentially from 5 to 11.

As a guide, when it is a matter of using the emulsion (Em) to convey a hydrophobic active material to the surface of a fabric present in a detergent bath, the pH of the outer aqueous phase (We) of the concentrated multiple emulsion (Em) may range from 5 to 11 and preferably from 6 to 8.

The outer phase (We), which is preferably aqueous, may also contain hydrophilic active materials, especially antibacterial agents, for instance methyl-chloroisothiazolinone and methylisothiazolinone (Kathon® CG sold by Rohm & Haas), and also buffers to regulate the pH, and additives for regulating the osmotic pressure, for instance salts (sodium chloride or sulfate, calcium chloride, etc.) or sugars (glucose) or polysaccharides (dextran, etc.).

The concentrations of salt, sugar and/or polysaccharide are such that the osmotic pressures of the outer (We) and inner (Wi) phases are equilibrated.

According to a first particular embodiment of the invention, the active material (A) contained in or constituting the hydrophobic phase (O) is a hydrophobic active material that is useful in the field of caring for or detergency of articles made of textile fibers, and the outer phase (We) is an aqueous liquid detergent formulation containing the dispersant and/or stabilizer (De) formed from a mixture of at least one nonionic hydrophilic surfactant and of at least one anionic hydrophilic surfactant, optionally combined with at least one nonionic hydrophilic (amphiphilic) polymer. The concentration of hydrophilic compound (De) in the aqueous outer phase (We) may range from 10% to 60% by weight and preferably from 15% to 50% by weight; its pH may range from 6 to 8. The mass ratio, expressed as solids, of nonionic surfactant/anionic surfactant may range from 3/1 to 1/1.

According to a second particular embodiment of the invention, the active material (A) is a hydrophobic active material that is useful in the field of caring for or detergency of articles made of textile fibers, and the outer phase (We) is a water-miscible nonaqueous liquid detergent formulation containing the dispersant and/or stabilizer (De) formed from a mixture of at least one nonionic hydrophilic surfactant and of at least one anionic hydrophilic surfactant, optionally combined with at least one nonionic hydrophilic (amphiphilic) polymer. The concentration of hydrophilic compound (De) in the nonaqueous outer phase (We) may range from 10% to 60% by weight and preferably from 15% to 50% by weight. The mass ratio, expressed as solids, of nonionic surfactant/anionic surfactant may range from 3/1 to 1/1.

According to a third particular embodiment of the invention, the active material (A) is a hydrophobic active material that is useful in the field of caring for articles made of textile fibers, and the outer phase (We) is an aqueous liquid rinsing formulation containing the dispersant and/or stabilizer (De) formed from at least one cationic hydrophilic surfactant and/or at least one cationic hydrophilic (amphiphilic) polymer, optionally as a mixture with at least one nonionic hydrophilic surfactant and/or at least one nonionic hydrophilic (amphiphilic) polymer. When a nonionic surfactant and/or a nonionic polymer is present, it may represent up to 50% of the weight of the cationic/nonionic mixture. The concentration of hydrophilic compound (De) in the aqueous outer phase (We) may range from 3.5% to 20% by weight; its pH may range from 2.5 to 6.

According to a fourth embodiment of the invention, the active material (A) is a hydrophobic active material that is useful in the field of paints, and the outer phase (We) is an aqueous paint formulation.

According to a fifth embodiment of the invention, the active material (A) is a hydrophobic active material that is useful in the field of body or hair makeup or care, and body, hair, hair dental or oral hygiene, and the outer phase (We) is a cosmetic aqueous formulation or a formulation for body, hair, dental or oral hygiene.

The multiple emulsion (Em) according to the invention may be obtained by using techniques involving only one reactor or two reactors.

A one-reactor technique may be performed by carrying out the following steps:
(a) the inverse emulsion (Ei) is prepared
(b) the outer phase containing the agent and/or stabilizer (De) is prepared
(c) the outer phase is introduced into the inverse emulsion (Ei) without stirring
(d) the mixture is stirred.

Step (a) of preparing the inverse emulsion (Ei) may be performed as described above.

Step (b) of preparing the outer phase (We) may be performed by mixing the constituent of the outer phase (We) (preferably water) and the dispersant and/or stabilizer (De).

The outer phase (We) may also comprise adjuvants such as preserving agents and osmotic pressure regulators.

The preparation of the outer phase may be performed at room temperature. However, it may be advantageous to prepare the outer phase (We) at a temperature in the region of that at which the inverse emulsion (Ei) is prepared.

Once the outer phase (We) has been obtained, it is added to the inverse emulsion (Ei), during step (c), without stirring.

Next, after all of the outer phase (We) has been introduced into the inverse emulsion (Ei), the mixture is stirred (step (d).

Advantageously, the stirring is performed using medium-shear mixers, for instance stirrers equipped with a frame paddle, mixers of planetary type, or alternatively those containing a mobile doctor blade and a paddle rotating in opposite directions (counter-stirring).

This stirring operation preferably takes place at a temperature at which the hydrophobic phase (O) is in a liquid form, and more particularly between 10 and 80° C.

The mean size of the globules of inner inverse emulsion (Ei) advantageously ranges between 1 and 100 μm, more particularly between 1 and 20 μm and advantageously between 5 and 15 μm. The mean globule size, corresponding to the median volume diameter (d50), which represents the diameter of the globule equal to 50% of the cumulative distribution, is measured with a Horiba machine and/or with an optical microscope.

The various constituents of the emulsion (Em) may be used in the amounts mentioned above.

When (We) is an aqueous phase, although the value of the pH of the aqueous phase is not limiting, it may be advantageous to adjust the pH of the outer aqueous phase by adding a base (sodium hydroxide or potassium hydroxide) or an acid (hydrochloric acid).

As an illustration, the usual pH range of the outer aqueous phase is between 0 and 14, preferably between 2 and 11 and more preferentially between 5 and 11.

After this stirring step (d), a concentrated multiple emulsion is obtained, the inverse emulsion/outer phase (We) weight ratio of which may range from 50/50 to 99/1, preferably from 70/30 to 98/2 and most particularly from 70/30 to 80/20.

A two-reactor technique may be performed by carrying out the following steps:
(a) the outer phase (We) containing the dispersant and/or stabilizer (De) is prepared as above
(b) the inverse emulsion (Ei) is prepared as above
(c) the inverse emulsion (Ei) is gradually introduced into the outer phase (We), with stirring.

Step (c) of preparing the actual multiple emulsion is performed with stirring; the stirring may be performed using a frame paddle. Typically, the stirring speed is relatively slow, of the order of 400 rpm.

The multiple emulsion (Em) obtained is similar to that obtained via the one-reactor technique.

Another two-reactor technique for preparing a similar multiple emulsion (Em) uses the following steps:
(a) the inverse emulsion (Ei) is prepared as above; the amount of inverse emulsion (Ei) prepared is divided into two parts
(b) the outer phase (We) containing the dispersant and/or stabilizer (De) is prepared
(c) the outer phase (We) is introduced into the first part of the inverse emulsion (Ei) without stirring
(d) the mixture is stirred
(e) the remainder of the inverse emulsion (Ei) is gradually introduced into the multiple emulsion obtained in step (d), with stirring.

When the dispersant and/or stabilizer (De) is made of at least one water-soluble or water-dispersible polysaccharide (PSA) constituting the stabilizer (Di), the multiple emulsion (Em) may be directly obtained by subjecting a mixture formed from the constituent(s) of the hydrophobic phase (O), the stabilizer (Di) and the constituent(s) of the outer phase (We) to a high-shear stirring operation.

Solid Form (Es)

The emulsion in solid form (Es) according to the invention may be obtained by adding, to the outer phase of the multiple emulsion (Em), a matrix (M)
made of a water-soluble or water-dispersible compound capable of forming, in the presence of the outer phase of the multiple emulsion (Em), a solid shell, after drying and optionally crystallizing
made of a water-soluble or water-dispersible solid material capable of adsorbing and/or absorbing the multiple emulsion (Em) and/or of becoming hydrated, while at the same time remaining in a solid form, or
made of an insoluble solid material capable of adsorbing and/or absorbing the multiple emulsion (Em).

The outer phase (We) of the multiple emulsion (Em) is preferentially aqueous.

Among the materials capable of forming the matrix (M), mention may be made of:
the following water-soluble or water-dispersible organic and inorganic compounds:
   polyethylene glycols (PEG) with a molecular mass of between 2000 and 100 000 g/mol
   copolymers of ethylenically unsaturated carboxylic acid or anhydride and of ethylenically unsaturated nonionic monomer
   water-soluble or water-dispersible polypeptides (PP) of natural or synthetic origin
   polyelectrolytes (PE) in acid form, belonging to the family of weak polyacids, with a molecular mass of less than 20 000 g/mol, preferably between 1000 and 5000 g/mol
   polyvinylpyrrolidones (PVP) with a molecular mass of less than 20 000 g/mol, preferably between 1000 and 10 000 g/mol
   polyvinyl alcohols (PVA) with a molecular mass of less than 100 000 g/mol and preferably having a degree of deacetylation of from 80 mol % to 99 mol % and preferably from 87 mol % to 95 mol %
   water-dispersible or water-soluble film-forming ampholytic polymers (AP)
   water-soluble or water-dispersible saccharides, osides or polyholosides (Oz)
   amino acids (AA) or water-soluble or water-dispersible salts of amino acids
   citric acid
   fatty acids
   urea
   surfactants (SA), the water-surfactant binary phase diagram of which comprises an isotropic phase that is fluid at 25° C. up to a concentration of at least 50% by weight of surfactant, followed by a rigid liquid crystal phase of hexagonal or cubic type at higher concentrations, which is stable at least up to 60° C.
   water-soluble or water-dispersible salts of alkali metals, for instance alkali metal silicates (Sil), carbonates (Carb), phosphates (Phos), sulfates, phosphonates, acetates, citrates and saturated or unsaturated fatty acid salts (stearates), and mixtures of sodium acetate and of citric acid, or
      mixtures thereof.
and also insoluble porous materials, for instance alkaline-earth metal carbonates or clays.

Among the copolymers of ethylenically unsaturated carboxylic acid or anhydride and of ethylenically unsaturated nonionic monomer, mention may be made of copolymers of an ethylenically unsaturated, linear or branched aliphatic, cycloaliphatic or aromatic monocarboxylic or polycarboxylic acid or anhydride and of α-monoolefins containing from 2 to 20 carbon atoms.

Among the acid or anhydride monomers, mention may be made of those containing from 3 to 10 carbon atoms, preferably those of formula
(R²′)(R²″)C═C(R²′″)COOH
in which R²′, (R²″) and R²′″ and are identical or different and represent
a hydrogen atom,
a hydrocarbon-based radical containing from 1 to 4 carbon atoms, preferably methyl,
a —COOH function,
a radical —R—COOH, in which R represents a hydrocarbon-based residue containing from 1 to 4 carbon atoms, preferably an alkylene residue containing 1 or 2 carbon atoms, most particularly methylene.

Preferentially, at least one of the radicals R²′ and R²″ is hydrogen.

By way of example, mention may be made especially of acrylic, methacrylic, crotonic, maleic, fumaric, citraconic or itaconic acid or anhydride.

Among the α-monoolefin monomers, mention may be made particularly of ethylene, propylene, 1-butene, isobutylene, n-1-pentene, 2-methyl-1-butene, n-1-hexene, 2-methyl-1-pentene, 4-methyl-1-pentene, 2-ethyl-1-butene, diisobutylene (or 2,4,4-trimethyl-1-pentene) and 2-methyl-3,3-dimethyl-1-pentene.

The mole ratio between the two types of monomer may preferentially range from 30/70 to 70/30.

Preferably, the copolymer of formula (i) is derived from the polymerization of maleic anhydride and of 2,4,4-trimethyl-1-pentene.

This type of copolymer is well known to those skilled in the art. Polymers of this type that may be mentioned include the product sold under the name Geropon® EGPM and T36 (maleic anhydride/diisobutylene) sold by Rhodia Chimie, and also Sokalan® CP9 (maleic anhydride/olefin) sold by BASF.

Among the water-soluble or water-dispersible synthetic polypeptides (PP), mention may be made of homopolymers or copolymers derived from the polycondensation of amino acids or of amino acid precursors, especially of aspartic acid and glutamic acid or precursors thereof, and hydrolysis. These polymers may be either homopolymers derived from aspartic acid or glutamic acid, or copolymers derived from aspartic acid and glutamic acid in any proportions, or copolymers derived from aspartic acid and/or glutamic acid and from other amino acids. Among the copolymerizable amino acids that may be mentioned are glycine, alanine, leucine, isoleucine, phenylalanine, methionine, histidine, proline, lysine, serine, threonine, cysteine, etc.

Among the polypeptides (PP) of plant origin, mention may be made of proteins of plant origin; these proteins are preferably hydrolyzed, with a degree of hydrolysis of less than or equal to 40%, for example from 5% to less than 40%.

Among the proteins of plant origin that may be mentioned, as a guide, are proteins derived from protein-yielding seeds, especially those of pea, horse bean, lupin, bean and lentil; proteins derived from cereal seeds, especially those of wheat, barley, rye, corn, rice, oat and millet; proteins derived from oil-yielding seeds, especially those of soybean, groundnut, sunflower, rapeseed and coconut; proteins derived from the leaves especially of alfalfa and nettle; and proteins derived from underground plant storage organs, especially that of potato and beetroot.

Among the proteins of plant origin that may be mentioned, for example, are muscle proteins, especially proteins of the stroma, and gelatin; proteins derived from milk, especially casein and lactoglobulin; and fish proteins.

The protein is preferably of plant origin and more particularly comes from soybean or wheat.

The polyelectrolyte (PE) may be chosen from those derived from the polymerization of monomers having the following general formula
(R¹)(R²)C═C(R³)COOH
in which formula R¹, R² and R³ are identical or different and represent
a hydrogen atom,
a hydrocarbon-based radical containing from 1 to 4 carbon atoms, preferably methyl,
a —COOH function,
a radical —R—COOH, in which R represents a hydrocarbon-based residue containing from 1 to 4 carbon atoms, preferably an alkylene residue containing 1 or 2 carbon atoms, most particularly methylene.

Nonlimiting examples that may be mentioned include acrylic acid, methacrylic acid, maleic acid, fumaric acid, itaconic acid and crotonic acid.

Copolymers that are also suitable are those obtained from the monomers corresponding to the above general formula and those obtained using these monomers and other monomers, in particular vinyl derivatives, for instance vinyl alcohols and copolymerizable amides, for instance acrylamide or methacrylamide. Mention may also be made of copolymers obtained from an alkyl vinyl ether and from maleic acid and also those obtained from vinylstyrene and from maleic acid, which are described especially in the Kirk-Othmer encyclopedia entitled “Encyclopedia of Chemical Technology”—Volume 18—3^{rd} edition—Wiley interscience publication—1982.

The preferred polyelectrolytes have a low degree of polymerization. The weight-average molecular mass of the polyelectrolytes is more particularly less than 20 000 g/mol. It is preferably between 1000 and 5000 g/mol.

An ampholytic polymer (AP) is a polymer comprising anionic or potentially anionic charges as a function of the pH and cationic or potentially cationic charges as a function of the pH, the potentially anionic or potentially cationic charges being taken into account for the calculation of the ratio of the total number of anionic charges to the total number of cationic charges.

The ampholytic film-forming polymer (AP) generally has a molecular mass of less than 500 000 g/mol, determined by aqueous gel-permeation chromatography (GPC).

The ampholytic film-forming polymer (AP) may be obtained from anionic and cationic ethylenically unsaturated monomers. It may also be obtained from a mixture of monomers also containing neutral monomers.

The anionic ethylenically unsaturated monomers may be chosen from acrylic, methacrylic, fumaric, maleic or itaconic acid or anhydride, N-methacryloylalanine, N-acryloylhydroxyglycine, etc. or water-soluble salts thereof; sulfonated or phosphonated water-soluble ethylenically unsaturated monomers, such as sulfopropyl acrylate or water-soluble salts thereof, water-soluble styrenesulfonates, vinylsulfonic acid and water-soluble salts thereof, or vinylphosphonic acid and water-soluble salts thereof.

The cationic ethylenically unsaturated monomers may be chosen from:
aminoacryloyl or acryloyloxy monomers, for instance trimethylaminopropyl methacrylate chloride, trimethylaminoethylacrylamide or -methacrylamide chloride or bromide, trimethylaminobutylacrylamide or -methacrylamide methyl sulfate, trimethylaminopropylmethacrylamide methyl sulfate (MES), (3-meth-acrylamidopropyl)trimethylammonium chloride (MAPTAC), (3-acrylamidopropyl)-trimethylammonium chloride (APTAC), methacryloyloxyethyltrimethylammonium chloride or methyl sulfate, and acryloyloxyethyltrimethylammonium chloride;
1-ethyl-2-vinylpyridinium bromide, chloride or methyl sulfate;
N,N-dialkyldiallylamine monomers, for instance N,N-dimethyldiallylammonium chloride (DADMAC);
quaternary monomers, for instance dimethylaminopropylmethacrylamide chloride, N-(3-chloro-2-hydroxypropyl)trimethylammonium chloride (DIQUAT), etc.

The ethylenically unsaturated neutral monomers may be chosen from acrylamide, N-isopropylacrylamide, N,N-dimethylacrylamide, dimethylaminoethyl methacrylate (DMAEMA), dimethylaminopropylmethacrylamide, vinyl alcohol, alkyl or hydroxyalkyl acrylates or methacrylates, polyoxyalkylene glycol acrylates or methacrylates, etc.

Ampholytic polymers (AP) that may be mentioned most particularly include the following copolymers or terpolymers:
MAPTAC/acrylic or methacrylic acid; DIQUAT/acrylic or methacrylic acid; DADMAC/acrylic or methacrylic acid;
MES/acrylic or methacrylic acid/DMAEMA;
MAPTAC/acrylic acid/acrylamide; MAPTAC/maleic anhydride/acrylamide; MAPTAC/vinylsulfonic acid/acrylamide;
DADMAC/acrylic acid/acrylamide; DADMAC/maleic anhydride/acrylamide; DADMAC/vinylsulfonic acid/acrylamide;
DIQUAT/acrylic acid/acrylamide; DIQUAT/maleic anhydride/acrylamide; DIQUAT/vinylsulfonic acid/acrylamide; with a ratio of the total number of anionic charges to the total number of cationic charges possibly ranging from 0.1 to 10.
   Among the saccharides (Oz) that may be mentioned are aldoses such as glucose, mannose, galactose and ribose, and ketoses such as fructose.

Osides are compounds resulting from the condensation, with elimination of water, of saccharide molecules with each other or alternatively of saccharide molecules with noncarbohydrate molecules. Among the preferred osides are holosides, which are formed by combining exclusively carbohydrate units and more particularly oligoholosides (or oligosaccharides) that comprise only a limited number of these units, i.e. a number in general of less than or equal to 10. Examples of oligoholosides that may be mentioned include sucrose, lactose, cellobiose, maltose and trehalose.

The water-soluble or water-dispersible polyholosides (or polysaccharides) are highly depolymerized; they are described, for example, in P. Arnaud's book entitled “Cours de Chimie Organique”, published by Gaultier-Villars, 1987. More particularly, these polyholosides have a weight-average molecular mass of less than 500 000 g/mol, preferably less than 20 000 g/mol.

Nonlimiting examples of polyholosides that may be mentioned include celluloses and cellulose derivatives (carboxymethylcellulose) and carrageenans; among the highly depolymerized polyholosides that may be mentioned are dextran, starch, xanthan gum and galactomannans such as guar or carob, these polysaccharides preferably having a melting point of greater than 100° C. and a solubility in water of between 5 and 500 g/l.

Among the amino acids (AA) that may be mentioned are monoamino monocarboxylic or dicarboxylic acids and diamino monocarboxylic acids, and water-soluble derivatives thereof.

The amino acids (AA) preferably have a side chain with acidobasic properties; they are chosen especially from arginine, lysine, histidine, aspartic acid, glutamic acid and hydroxyglutamic acid; they may also be in the form of derivatives, which are preferably water-soluble; they may be, for example, sodium, potassium or ammonium salts, for instance sodium glutamate, aspartate or hydroxyglutamate.

As regards the surfactants (SA) capable of constituting the matrix (M), the description of the fluid isotropic phase and of the rigid liquid-crystal phase of hexagonal or cubic type is given in R. G. Laughlin's book entitled “The Aqueous Phase Behavior of Surfactants”—Academic Press, 1994. Their identification by radiation (X-ray and neutron) scattering is described in V. Luzzati's book entitled “Biological Membranes, Physical Fact and Function”—Academic Press, 1968.

More particularly, the rigid liquid-crystal phase is stable up to a temperature at least equal to 55° C. The fluid isotropic phase can be poured, whereas the rigid liquid-crystal phase cannot.

Among the surfactants (SA) that may be mentioned are ionic glycolipid surfactants, especially derivatives of uronic acids (galacturonic acid, glucuronic acid, D-mannuronic acid, L-iduronic acid, L-guluronic acid, etc.), with a saturated or unsaturated, substituted or unsubstituted hydrocarbon-based chain containing from 6 to 24 carbon atoms and preferably from 8 to 16 carbon atoms, or salts thereof. Products of this type are described especially in patent application EP 532 370.

Other examples of surfactants (SA) are amphoteric surfactants such as amphoteric alkylpolyamine derivatives, for instance Amphionic XL@, Mirataine H2C-HA® sold by Rhône-Poulenc, and also Ampholac 7T/X® and Ampholac 7C/X® sold by Berol Nobel.

Among the alkali metal silicates (Sil), mention may be made especially of those with an SiO₂/M₂O mole ratio of from 1.6 to 3.5 with M representing a sodium or potassium atom.

Among the alkali metal or alkaline-earth metal phosphates (Phos), mention may be made especially of anhydrous sodium hexametaphosphate and anhydrous sodium tripolyphosphates.

Among the carbonates, mention may be made especially of those of sodium or of calcium.

The amount of matrix (M) used is such that it represents from 20% to 80% and preferably from 30% to 70% of the weight of the emulsion in solid form (Es) expressed as solids.

The presentation in solid form (Es) of the multiple emulsion (Em) may be prepared in different ways, depending on the nature of the matrix.

When it is a matrix that is meltable at a temperature below 80° C. and crystallizable, for instance polyethylene glycols with a molecular mass ranging from 2000 to 100 000 g/mol and preferably from 3000 to 50 000 g/mol, the multiple emulsion (Em) may be prepared in solid form by adding to said multiple emulsion (Em) polyethylene glycol melt at a temperature of about from 60 to 80° C., or as an aqueous solution, followed by crystallization by drying/cooling as a thin film in a ventilated oven, and flaking.

One variant of this process consists in preparing the multiple emulsion (Em) by dispersing the inverse emulsion (Ei) at a temperature of from 60 to 80° C. in the molten polyethylene glycol containing (De), followed by crystallization by drying/cooling as a thin film in a ventilated oven, and flaking.

When it is a matrix made of a material, preferably an inorganic material, which is anhydrous, porous and/or hydratable, capable of remaining in solid form after hydration, adsorption and/or absorption, for instance sodium tripolyphosphates or sodium carbonate, the presentation in solid form may be prepared by placing on a support on said material, optionally with moderate drying.

According to one preferential mode of preparation of the emulsion (E) in a solid form (Es), the outer aqueous phase of the multiple emulsion (Em) comprises at least one water-soluble or water-dispersible compound (M) that is not meltable at a temperature below 100° C., as additive for drying the multiple emulsion. Specifically, in the presence of these compounds, it becomes possible to dry the multiple emulsion (in other words, to remove the outer water from said emulsion) in order to obtain granules (or any other form).

Preferentially, the multiple emulsion (Em) is diluted with water preferably comprising the dispersant and/or stabilizer (De) and introduced into the matrix (M), and then dried.

The drying operation (consisting in removing the water of the outer aqueous phase) may be performed by any means known to those skilled in the art.

Preferably, the drying is performed such that at least 90% by weight of the outer aqueous phase is removed.

The drying may be performed in an oven, preferably as a thin film. Usually, the drying temperature is less than or equal to 100° C. More particularly, temperatures of between 50° C. and 90° C. are suitable for carrying out this method.

Another mode of drying of the emulsion is a “fast” method. Spray-drying, drying by use of Duprat® drums or lyophilization (freezing-sublimation) are suitable in this respect.

Spray-drying may be performed in a usual manner in any known apparatus, for instance a spraying tower combining spraying performed with a nozzle or a turbine with a stream of hot gas. The gas inlet temperature is from about 100° C. to 200° C. and the spraying gas outlet temperature is preferably between 55 and 100° C. These temperatures are given as a guide, and depend on the heat stability of the various components. Furthermore, it is defined according to the desired final water content in the granule.

Advantageously, the desired mean size of the granules (d50) is between 100 μm and a few millimeters (Sympatec), preferably between 100 and 800 μm. Such granules may be obtained directly using a twin-effect atomizer (drying/granulation). They may also be obtained using a single-effect atomizer (drying) combined with a granulating machine (fluidized bed) with spraying of water optionally containing material of the matrix (M).

In the case of multiple emulsion drying operations performed using a Duprat® drum or any means for rapidly obtaining a dry film that is separated from the drying support by scraping, for example, flakes or powder are obtained, which may optionally be ground. If necessary, these flakes may undergo a subsequent treatment, for instance an aggregation step, so as to obtain aggregated granules. Advantageously, the mean size (d50) of the granules obtained directly after drying is between 100 μm and a few millimeters (Sympatec) and preferably between 100 and 800 μm.

It should be noted that additives, such as anti-caking agents and fillers (for instance silica, kaolin, titanium dioxide, etc.) may be incorporated into the granules.

These additives may be introduced during the drying or as a post-addition.

A second subject of the invention consists of the use of an emulsion (E) comprising a liquid or meltable hydrophobic phase (O) containing and/or consisting of at least one hydrophobic active material (A), said emulsion (E) being:
in the form of a multiple emulsion (Em) comprising:
   an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di)
   an aqueous or water-miscible outer phase (We), in which is dispersed the inner emulsion (Ei), by means of at least one dispersant and/or stabilizer (De), or
in a solid form (Es), which is water-dispersible as a multiple emulsion (Em) in which the outer phase (We) is aqueous, comprising
   the inverse emulsion (Ei) dispersed in a water-soluble or water-dispersible solid matrix (M), and
   the dispersant and/or stabilizer (De) located at the interface of the inverse emulsion (Ei) and of the matrix (M) and optionally dispersed in the matrix (M)
      in which emulsion (E) the stabilizer (Di) at the interface of the two phases of the inner inverse emulsion (Ei) is made of a material chosen from water-soluble or water-dispersible polysaccharides (PSA), free of lipophilic polyorganosiloxane substituent groups:
the mean degree of polymerization (DP) of which is at least 1.5, preferably at least 20 ? and most particularly at least 100,
the Brookfield viscosity of which, at 25° C. as a solution at 1% by mass in water, is less than 20 000 mPa·s and preferably less than 5000 mPa·s, most particularly ranging from 1 to 4500 mPa·s;
   to convey, as an aqueous medium (B) in contact with a substrate (S), the hydrophobic phase (O) containing and/or consisting of at least one hydrophobic active material (A), to said substrate (S).

Said substrate (S) may be made of any material, especially of a metal or any natural, artificial or synthetic material, or of a mixture of these materials. Said hydrophobic phase (O) is preferably made of an active material capable of imparting its intrinsic properties or the benefits resulting therefrom to said substrate.

The aqueous outer phase (We) may itself constitute the aqueous medium (B), with release of the hydrophobic phase and of the hydrophobic active material by deposition or application and drying of the emulsion on the substrate (S).

The emulsion according to the invention may be used in paints, preferably water-based paints, or may itself constitute a paint, preferably a water-based paint, and may be used especially to convey a water-repellent agent onto a surface of the type such as a building material, plaster, cement, wood, etc., with release of the water-repellent agent by deposition and drying of the paint on the surface.

It may also be used for treating metals.

Similarly, it may be used in cosmetic compositions or may itself constitute an aqueous cosmetic composition (moisturizing creams, sun creams, makeup products, styling gels, etc.); the hydrophobic phase may be or may contain any hydrophobic active care material (for instance conditioning agents, disentangling agents, etc.), anti-UV agents, pigments, dyes, etc.

It may also be used to give surfaces made of a woven or nonwoven material of cellulose and/or synthetic origin, for body hygiene or household cleansing, intended to come into contact with the skin, for instance care, cleansing or makeup-removing wipes, absorbent papers (tissues), feminine protection (towels), diapers, etc., intrinsic benefits of the hydrophobic phase (O) and/or to the active materials (A) contained in the hydrophobic phase, and may do so during the preparation of said surfaces or as a post-treatment of said surfaces. Softening, antiodor, fragrancing, bactericidal, etc. properties may thus be imparted.

It may also be used during the manufacture or for the post-treatment of cardboard or cardboard packaging, to impart hydrophobic, antiodor, bactericidal, fragrancing, etc. properties.

The second subject of the invention consists in particular in the use of an emulsion (E) comprising a liquid or meltable hydrophobic phase (O) containing and/or consisting of at least one hydrophobic active material (A), said emulsion (E) being:
in the form of a multiple emulsion (Em) comprising:
   an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di)
   an aqueous or water-miscible outer phase (We), in which is dispersed the inner emulsion (Ei), using at least one dispersant and/or stabilizer (De), or
in a solid form (Es), which is water-dispersible as a multiple emulsion (Em) in which the outer phase (We) is aqueous, comprising
   the inverse emulsion (Ei) dispersed in a water-soluble or water-dispersible solid matrix (M), and
   the dispersant and/or stabilizer (De) located at the interface of the inverse emulsion (Ei) and of the matrix (M) and optionally dispersed in the matrix (M)
      in which emulsion (E) the stabilizer (Di) at the interface of the two phases of the inner inverse emulsion (Ei) is made of a material chosen from water-soluble or water-dispersible polysaccharides (PSA), free of lipophilic polyorganosiloxane substituent groups:
the mean degree of polymerization (DP) of which is at least 1.5, preferably at least 20 ? and most particularly at least 100,
the Brookfield viscosity of which, at 25° C. as a solution at 1% by mass in water, is less than 20 000 mPa·s and preferably less than 5000 mPa·s, most particularly ranging from 1 to 4500 mPa·s,
   to convey, in aqueous medium (B) placed in contact with a substrate (S), the hydrophobic phase (O) containing and/or consisting of at least one hydrophobic active material (A), to said substrate (S), the volume of said aqueous medium being sufficient to cause the destabilization and/or breaking of the emulsion (E) by dilution of said emulsion (E) and/or drying subsequent to the dilution of said emulsion (E), and the provision and/or release of the active material (A) contained in or constituting the hydrophobic phase (O), on the substrate (S).

Preferentially, the emulsion (E) is a multiple emulsion (Em) comprising at least 70% by weight of inner emulsion (Ei).

For good implementation of the invention, relative amounts of emulsion (Em) and of aqueous medium (B) equivalent to a dilution of from 2 to 100 times the volume of said emulsion (Em) may be used.

The emulsion according to the invention is particularly advantageous for conveying and depositing a hydrophobic active material onto a hydroxyapatite surface (tooth), a keratinous surface (skin, hair or scalp) or a textile surface.

When said substrate (S) is made of hydroxyapatite (teeth), the hydrophobic phase may contain hydrophobic agents with refreshing properties, agents for combating dental plaque, antiseptic agents, etc. The emulsion (E) may be included in or may itself form a composition for dental or oral hygiene, this composition being intended to be rinsed out or diluted. Such compositions may be toothpastes, mouthwashes, etc.

Said substrate (S) may especially be a keratinous surface, for instance the skin and the hair. The hydrophobic phase may be or may contain any hydrophobic active care material (for instance conditioning agents, disentangling agents, etc.), anti-UV agents, pigments, dyes, etc.; the emulsion (E) may be included in or may itself form a cosmetic composition intended to be rinsed off or diluted; it may especially be a shampoo, a hair conditioner, a shower gel, etc.

Said substrate (S) may be leather; the hydrophobic phase may be or may contain any hydrophobic active material capable of giving the substrate hydrophobicity, softness, suppleness, protection against external agents, etc.

Most preferentially, said substrate (S) is made of a textile material.

The textile substrate may be in the form of textile fibers or of articles made from natural textile fibers (cotton, flax or other natural cellulose-based material, wool, etc.), artificial textile fibers (viscose, rayon, etc.) or synthetic textile fibers (polyamide, polyester, etc.) or mixtures thereof.

Preferentially, said substrate is a textile surface made of a cellulose-based material, especially cotton.

Said hydrophobic phase (O) is preferably made of a textile care agent.

The particular examples of organosilicon materials (O1) and organic materials (O2) mentioned above are particularly suitable for use, most particularly organosilicon materials, especially amine-containing materials.

The benefits afforded by a lubricant hydrophobic phase (O1) or (O2) to a textile substrate are especially the provision of softness, anti-wrinkling, easy-ironing, abrasion resistance (protection especially against aging during the wearing of the item of clothing or the repeated washing operations), elasticity, color protection, fragrance-retaining, etc. properties.

Among the other active materials that afford other benefits in the field of caring for articles made of textile fibers, mention may be made in particular of fragrances; these fragrances are preferentially dissolved in the hydrophobic phase (O), especially made of organosilicon (O1) or organic (O2) materials.

The aqueous bath (B) in which the textile substrate is present in order to acquire benefits therefrom may be very varied. It may be, in a nonlimiting manner, a soaking, washing, rinsing, padding, etc. bath.

The emulsion (E) according to the invention may especially be used as an additive in a detergent composition for washing or rinsing articles made of textile fibers, or as a detergent or rinsing composition for washing or rinsing articles made of textile fibers, with the aim of conveying a hydrophobic care agent (textile care agent) and/or any other useful hydrophobic active material, and of promoting the deposition of said agent and/or said material onto an article made of textile fibers, especially of cotton, during the rinsing operation and/or during the drying operation subsequent to the main washing operation when it is a detergent washing composition, or during the subsequent drying operation when it is a rinsing composition.

Specifically, it has been found that the use of the emulsion (E) in the form of a multiple emulsion (Em) or in the form of a water-dispersible solid (Es) containing a hydrophobic care phase (O), as a detergent composition or in a detergent composition for washing laundry in a washing machine, used in the wash cycle, and without the addition of softening rinsing liquid during the rinse cycle, makes it possible to give the washed laundry softness, suppleness, anti-wrinkling, easy-ironing, abrasion resistance, elasticity, color-protection, fragrance-retaining, etc. properties.

It has also been found that the use of the emulsion (E) in the form of a multiple emulsion (Em) or in the form of a water-dispersible solid (Es) containing a hydrophobic care phase (O), as a rinsing composition or in a composition for rinsing laundry, can give the laundry, after drying, softness, suppleness, anti-wrinkling, easy-ironing, abrasion resistance, elasticity, color-protection, fragrance-retaining, etc. properties.

The deposition of the hydrophobic phase (O) containing or consisting of the active material (A) onto the substrate may be a deposition by adsorption, co-crystallization, trapping and/or adhesion.

The amount of emulsion (E) in the form of a multiple emulsion (Em) or in the form of a water-dispersible solid (Es) that may be present in a composition for washing articles made of textile fibers, according to the third subject of the invention, corresponds to an amount of hydrophobic phase (O) representing from 0.0001% to 25% and preferably from 0.0001% to 5% of the total weight of the composition, with relative amounts of emulsion, expressed as multiple emulsion (Em), and of aqueous medium (B) equivalent to a 2-fold to 100-fold dilution of the volume of said emulsion (Em).

The amount of emulsion (E) in the form of a multiple emulsion (Em) that may be present in a composition for rinsing articles made of textile fibers, according to the third subject of the invention, corresponds to an amount of hydrophobic phase (O) representing from 0.0001% to 25% and preferably from 0.0001% to 5% of the total weight of the composition, with relative amounts of emulsion, expressed as multiple emulsion (Em), and of aqueous medium (B) equivalent to a 2-fold to 100-fold dilution of the volume of said emulsion (Em).

A washing composition as a compacted or noncompacted powder, or as a liquid, for articles made of textile fibers may contain at least one surfactant preferably chosen from anionic and nonionic surfactants, or mixtures thereof. Among the anionic surfactants, mention may be made of (C₈-C₁₅)alkyl benzene-sulfonates (in a proportion of 0-30%, preferably 1-25% and more preferentially 2-15% by weight).

In addition, mention may be made of primary or secondary alkyl sulfates, in particular primary (C₈-C₁₅)alkyl sulfates; alkyl ether sulfates; olefin sulfonates; alkyl xylenesulfonates; dialkyl sulfosuccinates; fatty acid ester sulfonates; the sodium salts are generally preferred.

Among the nonionic surfactants that may be mentioned are primary or secondary alcohol ethoxylates, in particular C₈-C₂₀ aliphatic alcohol ethoxylates containing from 1 to 20 mol of ethylene oxide per mole of alcohol, and more particularly C₁₀-C₁₅ primary or secondary aliphatic alcohol ethoxylates containing from 1 to 10 mol of ethylene oxide per mole of alcohol; nonethoxylated nonionic surfactants, for instance alkylpolyglucosides, glycerol monoethers and polyhydroxyamides (glucamides) may also be mentioned.

Preferably, the content of nonionic surfactants is 0-30%, preferably 1-25% and more preferentially 2-15% by weight.

The choice and amount of surfactant depends on the intended use of the detergent composition. The surfactant systems to be chosen for washing textiles by hand or in a machine are well known to formulators.

Amounts of surfactants as high as 60% by weight may be present in handwashing compositions. Amounts of 5-40% by weight are generally suitable for washing textiles in a machine. Typically, these compositions comprise at least 2% by weight, preferably 2-60%, more preferentially 15-40% and particularly 25-35% by weight. It is also possible to include cationic monoalkyl surfactants. Mention may be made of the quaternary ammonium salts of formula R¹R²R³R⁴N⁺X⁻ in which the groups R are long or short hydrocarbon-based alkyl, hydroxyalkyl or ethoxylated alkyl chains, X being a counterion (R¹ is a C₈-C₂₂, preferably a C₈-C₁₀, or a C₁₂-C₁₄ alkyl group and R² is a methyl group, R³ and R⁴, which may be identical or different, being a methyl or hydroxymethyl group); and also cationic esters, for instance choline esters.

The detergent compositions for the majority of washing machines generally contain an anionic surfactant other than soaps, or a nonionic surfactant, or mixtures thereof, and optionally a soap.

The detergent compositions for washing textiles generally contain at least one detergence adjuvant (“builder”); the total amount of detergence adjuvant is typically 5-80% and preferably 10-60% by weight.

Mention may be made of inorganic adjuvants, for instance sodium carbonate, crystalline or amorphous aluminosilicates (10-70% and preferably 25-50% as solids), lamellar silicates, inorganic phosphates (sodium orthophosphate, pyro-phosphate and tripolyphosphate). Further details relating to aluminosilicates and zeolites that are particularly suitable are given in WO 03/020819.

Mention may also be made of organic detergence adjuvants, for instance polymers of the type such as polyacrylates, acrylic/maleic copolymers and acrylic phosphinates; polycarboxylate monomers, for instance citrates, gluconates, oxydisuccinates, glyceryl mono-, di- and trisuccinates, dipicolinates, hydroxyethyl-aminodiacetates, malonates or succinates of alkyl or of alkenyl; sulfonated fatty acid salts, etc.

Preferably, the organic detergence adjuvants are citrates (5-30% and preferably 10-25% by weight) or acrylic polymers, more particularly acrylic/maleic copolymers (0.5-10% and preferably 1-10% by weight).

When they are made of compacted or noncompacted powder, the compositions may favorably contain a bleaching system, especially peroxide compounds, for instance inorganic persalts (perborates, percarbonates, perphosphates, persilicates and persulfates, preferably sodium perborate monohydrate or tetrahydrate, and sodium percarbonate) or organic peroxy acids (urea peroxide), capable of releasing oxygen in solution.

The peroxide bleaching compound is favorably present in a proportion of 0.1-35% and preferably 0.5-25% by weight. It may be combined with a bleaching activator to improve the bleaching at low temperature. It is favorably present in an amount of 0.1-8% and preferably 0.5-5% by weight. The preferred activators are peroxycarboxylic acids, especially peracetic and pernonanoic acids. Mention may be made most particularly of N,N,N′,N′-tetraacetylethylenediamine (TAED) and sodium nonanoyloxybenzenesulfonate (SNOBS).

The compositions also generally comprise one or more enzymes, especially proteases, amylases, cellulases, oxidases, peroxidases and lipases (0.1-3% by weight), fragrances, anti-redeposition agents, anti-soiling agents, color-transfer counteractants, nonionic softeners, etc.

The detergent compositions for washing textiles may also be in the form of nonaqueous liquid tablets in an envelope made of a material that disperses in the detergent medium, for instance polyvinyl alcohol.

They comprise at least one water miscible alcohol such as, especially, isopropyl alcohol, in an amount that may range from 5% to 20% by weight.

They may contain at least one surfactant preferably chosen from anionic and nonionic surfactants, or mixtures thereof, in an amount that may range from 20% to 75% by weight.

They may also comprise organic detergence adjuvants (“builders”), for instance sodium citrates, phosphonates, etc., in an amount that may range from 5% to 20% by weight; they may also comprise fragrances, dyes, etc.

The compositions for rinsing articles made of textile fibers may contain cationic or nonionic softeners. They may represent from 0.5% to 35%, preferably 1-30% and more preferentially 3-25% of the weight of the rinsing composition.

The cationic softeners are substantially water-insoluble quaternary ammonium compounds, comprising a single alkyl or alkenyl chain containing at least 20 carbon atoms, or preferably compounds containing at least two polar heads and two alkyl or alkenyl chains containing at least 14 carbon atoms. Most preferentially, the softening compounds contain two alkyl or alkenyl chains containing at least 16 carbon atoms, and particularly at least 50% of the alkyl or alkenyl groups contain 18 or more carbon atoms. Most preferentially, linear alkyl or alkenyl chains are predominant.

In the commercial softening rinsing formulations, quaternary ammonium compounds containing two long aliphatic chains are very commonly used, for instance distearyldimethylammonium chloride or ditallowalkyldimethylammonium chloride. The rinsing compositions may also comprise nonionic softeners, for instance lanolin; lecithins and other phospholipids are also suitable. The rinsing compositions may also contain nonionic stabilizers, for instance alkoxylated C₈-C₂₂ linear alcohols containing from 10 to 20 mol of alkylene oxide, C₁₀-C₂₀ alcohols, and mixtures thereof. The amount of nonionic stabilizer represents 0.1-10%, preferably 0.5-5% and most particularly 1-4% of the weight of the composition. The mole ratio of the quaternary ammonium compound and/or other softening cationic agent to the stabilizer is favorably 40/1-1/1 and preferably 18/1-3/1.

The composition may also comprise fatty acids, especially monocarboxylated (C₈-C₂₄) alkyl or alkenyl acids or polymers thereof; they are preferably saturated and unsaponified, for instance oleic acid, lauric acid or tallow acid. They may be used in a proportion of at least 0.1% and preferably of at least 0.2% by weight. In the concentrated compositions, they may be present in a proportion of 0.5-20% and preferably 1-10% by weight. The mole ratio of the quaternary ammonium compound and/or other softening cationic agent to the fatty acid is favorably 10/1-1/10.

A final subject of the invention consists of a process for conveying, to a substrate (S) in contact with an aqueous medium (B), at least one active material contained in or constituting a liquid or meltable hydrophobic phase (O) of an emulsion (E), said emulsion being:
in the form of a multiple emulsion (Em) comprising:
   an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di)
   an aqueous or water-miscible outer phase (We), in which is dispersed the inner emulsion (Ei), using at least one dispersant and/or stabilizer (De), or
in a solid form (Es), which is water-dispersible as a multiple emulsion (Em), comprising
   the inverse emulsion (Ei) dispersed in a water-soluble or water-dispersible solid matrix (M), and
   the dispersant and/or stabilizer (De) located at the interface of the inverse emulsion (Ei) and of the matrix (M) and optionally dispersed in the matrix (M)
      in which emulsion (E) the stabilizer (Di) at the interface of the two phases of the inner inverse emulsion (Ei) is made of a material chosen from water-soluble or water-dispersible polysaccharides (PSA), free of lipophilic polyorganosiloxane substituent groups:
the mean degree of polymerization (DP) of which is at least 1.5, preferably at least 20 ? and most particularly at least 100,
the Brookfield viscosity of which, at 25° C. as a solution at 1% by mass in water, is less than 20 000 mPa·s and preferably less than 5000 mPa·s, most particularly ranging from 1 to 4500 mPa·s,
   by placing the emulsion (E) with the substrate (S) in contact with the aqueous medium (B).

The final subject of the invention is directed in particular toward a process for conveying, to a substrate (S) in contact with an aqueous medium (B), at least one active material contained in or constituting a liquid or meltable hydrophobic phase (O) of an emulsion (E), said emulsion being:
in the form of a multiple emulsion (Em) comprising:
   an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di)
   an aqueous or water-miscible outer phase (We), in which is dispersed the inner emulsion (Ei), using at least one dispersant and/or stabilizer (De), or
in a solid form (Es), which is water-dispersible as a multiple emulsion (Em), comprising
   the inverse emulsion (Ei) dispersed in a water-soluble or water-dispersible solid matrix (M), and
   the dispersant and/or stabilizer (De) located at the interface of the inverse emulsion (Ei) and of the matrix (M) and optionally dispersed in the matrix (M)
      in which emulsion (E) the stabilizer (Di) at the interface of the two phases of the inner inverse emulsion (Ei) is made of a material chosen from water-soluble or water-dispersible polysaccharides (PSA), free of lipophilic polyorganosiloxane substituent groups:
the mean degree of polymerization (DP) of which is at least 1.5, preferably at least 20 and most particularly at least 100,
the Brookfield viscosity of which, at 25° C. as a solution at 1% by mass in water, is less than 20 000 mPa·s and preferably less than 5000 mPa·s, most particularly ranging from 1 to 4500 mPa·s,
   by placing the emulsion (E) with the substrate (S) in contact with the aqueous medium (B), the volume of said aqueous medium (B) being sufficient to cause the destabilization and/or breaking of the emulsion (E) by dilution of said emulsion (E) and/or drying subsequent to the dilution of said emulsion (E), and the provision and/or release of the active material (A) contained in or constituting the hydrophobic phase (O), on the substrate (S).

Preferentially, the emulsion (E) is a multiple emulsion (Em) comprising at least 70% by weight of inner emulsion (Ei).

The conditions for performing said process have already been described above.

The examples that follow are given as a guide.

The following are used in the examples that follow
As hydrophobic phase (O) and active material (A), the aminosilicone oil Rhodorsil® Extrasoft sold by Rhodia;
As stabilizer (Di)
   the depolymerized guar Meypro-GAT® 7 sold by Rhodia with a weight-average molecular mass of about 47 000, a degree of polymerization of about 300, and a Brookfield viscosity of 1-5 mPa·s at 1% in water at 25° C.;
   a cellulose monoacetate with a degree of polymerization of about 300, a degree of substitution of 0.67 and a Brookfield viscosity of 1-5 mPa·s at 1% in water at 25° C.;
As dispersant and/or stabilizer (De), Arlatone® F 127G, of formula HO(CH₂CH₂O)ₓ(CH(CH₃)CH₂O)_{y}(CH₂CH₂O)_{z}H sold by ICI -Uniqema

### EXAMPLE 1

### Multiple Emulsion

Composition of the Inverse Emulsion (Ei):

50% by weight of inner aqueous phase consisting of:

| | |
|---|---|
| Water | 90 parts by weight |
| Meypro-GAT ® 7 | 10 parts by weight |

50% by weight of aminosilicone oil phase (O)

| | |
|---|---|
| Rhodorsil ® Extrasoft oil | 100 parts by weight |

Preparation of the Inverse Emulsion (Ei):
Preparation of the Inner Aqueous Phase

The water is introduced into a 1 L reactor equipped with a frame-paddle stirrer (diameter 54 mm, speed 400 rpm), at room temperature.

The Meypro-GAT® 7 powder is then introduced gradually with stirring, at room temperature.

The mixture is stirred for two hours at room temperature so as to disperse the particles of Meypro-GAT® 7 gel homogeneously. The dispersion may optionally be sheared using a machine of rotor-stator type (Ultra-Turrax GT45 type).

The pH of the inner aqueous solution/dispersion is from 5 to 7.5.

Preparation of the Inverse Emulsion

The Rhodorsil® Extrasoft oil is introduced into a 2 L reactor equipped with a frame-paddle stirrer (diameter 90 mm, speed 400 rpm).

The inner aqueous phase is then introduced, over 45 minutes, at room temperature. Stirring is continued for 15 minutes to refine the emulsion.

An inverse emulsion (Ei) is obtained, the drops of dispersed aqueous phase (Wi) of which have a particle size of 1 μm (observation made by optical microscopy on a sample without and with predilution in the Extrasoft oil).

Composition of the Multiple Emulsion:

91% by weight of above inverse emulsion (Ei)
9% by weight of outer aqueous phase (We), obtained from 9 parts by weight of an aqueous solution containing 10% by weight of Arlatone F 127G®, which corresponds to
   10% Arlatone® F 127G dry
   90% water.
      Preparation of the multiple emulsion:
      Preparation of the Outer Aqueous Phase

The water is introduced into a 0.5 L beaker equipped with a magnetic-bar stirrer, and the Arlatone F 127G powder is gradually introduced with stirring, at room temperature.

The mixture is stirred for 15 minutes at room temperature, so as to obtain a homogeneous solution (solution containing 10% Arlatone F 127G).

Preparation of the Double Emulsion

The outer aqueous phase prepared above is added, without stirring, and rapidly at room temperature, to the inverse emulsion (Ei) obtained above contained in the 2 L reactor equipped with a frame-paddle stirrer (diameter 90 mm).

The mixture is then stirred (frame-paddle stirring—diameter 90 mm, speed 200 rpm). A multiple emulsion is rapidly obtained; this emulsion is kept stirring for 10 minutes to refine it.

A concentrated multiple emulsion (Em) is obtained, which is in the form of a nonpourable viscous cream; the mean size of the droplets (d50) of the inner inverse emulsion is in the region of 10 μm, with a low polydispersity index.

### EXAMPLE 2

### Multiple Emulsion

A multiple emulsion is prepared according to the procedure of example 1, replacing the 10 parts by weight of Meypro-GAT® 7 with 10 parts of cellulose monoacetate.

### EXAMPLE 3

### Deposition of Rhodorsil® Extrasoft Oil Onto Cotton in a Detergent Bath

Test

The test is performed in a laboratory Tergotometer, which is well known to detergent composition formulators. The machine simulates the mechanical and thermal effects of American pulse-type washing machines; by virtue of the presence of 6 washing drums, it allows series of simultaneous tests to be performed with an appreciable saving in time.

Preparation

The drums are heated to 40° C.

500 ml of water with a hardness of 30°HT (diluted Contrexeville® mineral water) are introduced into each drum.

5 g of a detergent powder composition for machine-washing laundry are introduced into each drum (i.e. 10 g/l).

The Tergotometer is switched on (100 cycles/minute for 5 minutes) to dissolve the detergent product.

Wash Cycle

An amount of double emulsion prepared in example 1 or example 2, corresponding to 0.5 g of Rhodorsil® Extrasoft oil, is introduced into each drum. Three terry-toweling cotton test pieces (10 cm×10 cm) are introduced into each drum. The test pieces are washed at 100 cycles/minute for 20 minutes.

Rinse Cycle

The fabric squares are then rinsed 3 times for 5 minutes (each time) with cold water.

Drying

The fabric squares are taken out and air-dried.

An estimation of the softness performance is given by a panel of trained experts, in a blind test.

The performance obtained is given in the table that follows (“Emulsion”) and compared with that obtained with:
washing in a Tergotometer without addition of the multiple emulsion of example 1 or 2 (“Reference 1”)
washing in a Tergotometer without addition of the multiple emulsion of example 1 or 2, followed by rinsing with addition of 5 g of a commercial softening formulation on the third rinse (“Reference 2”).

The results obtained are given in the following table:

| | Formulation | | |
|---|---|---|---|
| | | | Emulsion of |
| | Reference 1 | Reference 2 | example 1 |
| Softness | 3% | 42% | 55% |
| preference | | | |
| | Formulation | | |
| | | | Emulsion of |
| | Reference 1 | Reference 2 | example 2 |
| Softness | 4% | 39% | 57% |
| preference | | | |

1-45. (canceled)

46) An emulsion (E) comprising a liquid or meltable hydrophobic phase (O) containing and/or consisting of at least one hydrophobic active material (A), said emulsion (E) being:
in the form of a multiple emulsion (Em) comprising:
an inner inverse emulsion (Ei) comprising said continuous liquid or meltable hydrophobic phase (O), an aqueous dispersed phase (Wi) and, at the interface of the two phases, at least one water-soluble or water-dispersible stabilizer (Di) an aqueous or water-miscible outer phase (We), in which is dispersed the inner emulsion (Ei), by means of at least one dispersant and/or stabilizer (De), or
in a solid form (Es), which is water-dispersible as a multiple emulsion (Em) in which the outer phase (We) is aqueous, comprising
the inverse emulsion (Ei) dispersed in a water-soluble or water-dispersible solid matrix (M), and
the dispersant and/or stabilizer (De) located at the interface of the inverse emulsion (Ei) and of the matrix (M) and optionally dispersed in the matrix (M),
said emulsion having the stabilizer (Di) at the interface of the two phases of the inner inverse emulsion (Ei) made of a water-soluble or water-dispersible polysaccharides (PSA), whose mean degree of polymerization (DP) is at least 1.5, preferably at least 20 and most particularly at least 100, and the Brookfield viscosity, at 25° C. as a solution at 1% by mass in water, is less than 20 000 mPa·s and optionally, ranging from 1 to 4500 mPa·s, and
said polysaccharides (PSA) also being free of lipophilic polyorganosiloxane substituent groups.

47) The emulsion as claimed in claim 46), wherein the hydrophobic phase (O) is made of at least one organic or organosilicon material or of a mixture of at least one organic material and of at least one organosilicon material, which is liquid or meltable and insoluble in an aqueous phase.

48) The emulsion as claimed in claim 46), wherein the active material (A) is made of at least one organic or organosilicon material, of a mixture of at least one organic material and of at least one organosilicon material, which is liquid or meltable and insoluble in an aqueous phase, of at least one solid or liquid inorganic material that is insoluble in an aqueous phase or of a mixture of at least one of said inorganic materials and of at least one of said organic materials and/or of at least one of said organosilicon materials.

49) The emulsion as claimed in claim 46), wherein said hydrophobic phase (O) and/or said active material (A) is an oil, a wax or a resin made of a linear, cyclic, branched or crosslinked polyorganosiloxane.

50) The emulsion as claimed in claim 49), wherein said polyorganosiloxane is a nonionic or amino polyorganosiloxane.

51) The emulsion as claimed in claim 46), wherein said hydrophobic phase (O) and/or said active material (A) is made of an organic material selected from the group consisting of: C₁-C₃₀ carboxylic acid mono-, di- or triglycerides or mixtures thereof, plant oils, technical oils, especially cooked, blown or standolized linseed oils, sucroesters, sucroglycerides, C₁-C₃₀ alcohol esters of C₁-C₃₀ carboxylic or C₂-C₃₀ dicarboxylic acids, ethylene glycol or propylene glycol monoesters or diesters of C₁-C₃₀ carboxylic acids, propylene glycol C₄-C₂₀ alkyl ethers, di (C₈-C₃₀) alkyl ethers, mineral oils, naphtha oils, liquid paraffins, polybutenes, organic waxes having alkyl chains containing from 4 to 40 carbon atoms, animal waxes, plant waxes, mineral waxes, hydrocarbon-based waxes having from 4 to 35 carbon atoms, and synthetic waxes.

52) The emulsion as claimed in claim 46), wherein the active material (A) is contained in the hydrophobic phase (O) and is made of one or more fragrancing molecules, an organic or organosilicon UV stabilizer, a hydrophobic bactericidal agent, solid polyamide capsules, silica particles or particles of another inorganic oxide or compound.

53) The emulsion as claimed in claim 46), wherein said polysaccharide (PSA) or its skeleton is a linear or branched, nonionic or ionic homopolysaccharide or heteropolysaccharide, having identical or different glycosyl units linked via β(1-4) bonds, and optionally, β(1-3) and/or β(1-6) bonds.

54) The emulsion as claimed in claim 53), wherein the hydroxyl functions of the glycosyl units are substituted and/or modified with nonionic or ionic groups other than lipophilic polyorganosiloxane groups.

55) The emulsion as claimed in claim 53), wherein said polysaccharide (PSA) is selected from the group consisting of depolymerized galactomannans, optionally modified or substituted with nonionic groups; cellulose monoacetates with a degree of substitution of from 0.3 to less than 1.2, hydroxypropylcelluloses with a degree of modification of about from 0.2 to 1.5, carboxymethylcelluloses with a degree of substitution of from 0.05 to 1.2; dextrins optionally containing hydroxyethyl, hydroxypropyl or quaternized aminoalkyl groups, xyloglycans, arabinoxylans, and alkylpolyglycosides.

56) The emulsion as claimed in claim 46), wherein the dispersed aqueous phase (Wi) presents a mass ratio of the mass of the dispersed aqueous phase (Wi) to the hydrophobic phase (O) ranging from 5/95 to 95/5 and optionally from 30/70 to 80/20.

57) The emulsion as claimed in claim 46), wherein the stabilizer (Di) presents a ratio of the mass of stabilizer (Di) to the mass of hydrophobic phase (O) ranges from 0.1/100 to 500/100, optionally from 0.5/100 to 50/100.

58) The emulsion as claimed in claim 46), wherein said dispersant and/or stabilizer (De) is a hydrophilic surfactant, a hydrophilic polymer or a hydrophilic amphiphilic polymer.

59) The emulsion as claimed in claim 58), wherein said dispersant and/or stabilizer (De) is formed from
(a) at least one nonionic hydrophilic surfactant
(b) at least one anionic hydrophilic surfactant
(c) at least one cationic hydrophilic surfactant
(d) at least one nonionic hydrophilic polymer
(e) at least one nonionic hydrophilic amphiphilic polymer
(f) at least one anionic hydrophilic polymer
(g) at least one anionic hydrophilic amphiphilic polymer
(h) at least one cationic hydrophilic polymer
(i) at least one cationic hydrophilic amphiphilic polymer
(j) or a mixture of at least two of said surfactants and/or polymers (a) to (d) above, which are compatible.

60) The emulsion as claimed in claim 58), wherein the surfactant(s) and/or polymer(s) (De) present in the outer phase (We) has a total content of between 0.01% and 50% by weight, preferably between 0.1% and 10% and more particularly between 0.5% and 5% by weight relative to the inverse emulsion (Ei).

61) The emulsion as claimed in claim 58), wherein said hydrophilic polymer (De) is made of or comprises at least one water-soluble or water-dispersible polysaccharide (PSA) (Di).

62) The emulsion as claimed in claim 46), wherein the inverse emulsion (Ei) and outer phase (We) comprising the dispersant and/or stabilizer (De), present a mass ratio of inner inverse emulsion (Ei)/outer phase (We) comprising the dispersant and/or stabilizer (De), ranging from 50/50 to 99/1, optionally from 70/30 to 80/20.

63) The emulsion as claimed in claim 1) to 17), wherein the dispersant and/or stabilizer (De) and the inner inverse emulsion (Ei) present a mass ratio, expressed as solids, of dispersant and/or stabilizer (De)/mass of the inner inverse emulsion (Ei), ranging from 0.01/100 to 50/100, optionally from 0.5/100 to 5/100.

64) The emulsion as claimed in claim 46), wherein the dispersant and/or stabilizer (De) present concentration in the outer phase (We), ranging from 1% to 50%.

65) The emulsion as claimed in claim 46), wherein the outer phase (We) is an aqueous phase.

66) The emulsion as claimed in claim 46), wherein the outer phase (We) is an alcoholic or aqueous-alcoholic phase, optionally isopropanol or ethanol.

67) The emulsion as claimed in claim 46), wherein the active material (A) contained in or constituting the hydrophobic phase (O) is a care or detergence agent for articles made of textile fibers, and the outer phase (We) is an aqueous liquid detergent formulation containing the dispersant and/or stabilizer (De) formed from a mixture of at least one nonionic hydrophilic surfactant and of at least one anionic hydrophilic surfactant, optionally combined with at least one nonionic hydrophilic (amphiphilic) polymer.

68) The emulsion as claimed in claim 46), wherein the active material (A) contained in or constituting the hydrophobic phase (O) is a care or detergence agent for articles made of textile fibers, and the outer phase (We) is a water-miscible nonaqueous liquid detergent formulation, containing the dispersant and/or stabilizer (De) formed from a mixture of at least one nonionic hydrophilic surfactant and of at least one anionic hydrophilic surfactant, optionally combined with at least one nonionic hydrophilic (amphiphilic) polymer.

69) The emulsion as claimed in claim 46), wherein the active material (A) contained in or constituting the hydrophobic phase (O) is a care agent for articles made of textile fibers, and the outer phase (We) is an aqueous liquid rinsing formulation, containing the dispersant and/or stabilizer (De) formed from at least one cationic hydrophilic surfactant and/or from at least one cationic hydrophilic (amphiphilic) polymer, optionally mixed with at least one nonionic hydrophilic surfactant and/or at least one nonionic hydrophilic (amphiphilic) polymer.

70) The emulsion as claimed in claim 46), wherein the active material (A) contained in or constituting the hydrophobic phase (O) is an agent in the field of paints, and in that the outer phase (We) is a water-based paint.

71) The emulsion as claimed in claim 46), wherein the active material (A) contained in or constituting the hydrophobic phase (O) is an agent in the field of cosmetics or body care, and the outer phase (We) is an aqueous cosmetic composition.

72) The emulsion as claimed in claim 46), being in (Es) form and the solid matrix (M) is made of:
polyethylene glycols with a molecular mass of between 2000 and 100 000 g/mol, copolymers of ethylenically unsaturated carboxylic acid or anhydride and of ethylenically unsaturated nonionic monomer, water-soluble or water-dispersible polypeptides of natural or synthetic origin, polyelectrolytes in acid form, belonging to the family of weak polyacids, with a molecular mass of less than 20 000 g/mol, optionally between 1000 and 5000 g/mol, polyvinylpyrrolidones with a molecular mass of less than 20 000 g/mol and preferably from 1000 to 10 000 g/mol, polyvinyl alcohols with a molecular mass of less than 100 000 g/mol, optionally having a degree of deacetylation of from 80 mol % to 99 mol %, water-soluble or water-dispersible film-forming ampholytic polymers, water-soluble or water-dispersible saccharides, osides or polyholosides, water-soluble or water-dispersible amino acids or amino acid salts, citric acid, fatty acids, urea, surfactants whose water-surfactant binary phase diagram comprises an isotropic phase that is fluid at 25° C. up to a concentration of at least 50% by weight of surfactant, followed by a rigid liquid crystal phase of hexagonal or cubic type at higher concentrations, which is stable at least up to 60° C., water-soluble or water-dispersible alkali metal salts, alkali metal salts of saturated or unsaturated fatty acids, or mixtures of sodium acetate and of citric acid.

73) A process for conveying, to a substrate (S) in contact with an aqueous medium (B), at least one active material (A) contained in or constituting a liquid or meltable hydrophobic phase (O) of the emulsion (E) as defined in claim 46), by placing said emulsion (E) with the substrate (S) in contact with the aqueous medium (B).

74) The process as claimed in claim 73), wherein said aqueous medium (B) has a volume sufficient to cause the destabilization and/or breaking of the emulsion (E) by dilution of said emulsion (E) and/or drying subsequent to the dilution of said emulsion (E), and the provision and/or release of the active material (A) contained in or constituting the hydrophobic phase (O), on the substrate (S).

75) The process as claimed in claim 74), wherein the emulsion (E) is a multiple emulsion (Em) comprising at least 70% by weight of inner emulsion (Ei).
